(19) United States
(12) Patent Application Publication
(10) Pub. No.: US 2025/0029676 A1
(43) Pub. Date: Jan. 23, 2025

(54) PROFILING EPIGENETIC AGE IN SINGLE CELLS AND WITH LOW-PASS SEQUENCING DATA

(71) Applicant: The Brigham and Women's Hospital, Inc., Boston, MA (US)

(72) Inventors: Alexandre TRAPP, Boston, MA (US); Vadim GLADYSHEV, Boston, MA (US); Csaba KEREPESI, Boston, MA (US)

(21) Appl. No.: 18/281,025

(22) PCT Filed: Mar. 14, 2022
(86) PCT No.: PCT/US2022/020222

§ 371 Date: Sep. 8, 2023

Related U.S. Application Data
(60) Provisional application No. 63/229,167, filed on Aug. 4, 2021.
(60) Provisional application No. 63/160,246, filed on Mar. 12, 2021.

(51) Int. Cl. G16B 20/00

(52) U.S. Cl. CPC G16B 20/00

(57) ABSTRACT

The invention features a method of estimating an epigenetic age of a single cell from a mammalian tissue, the method comprising: creating a reference methylation probability data set comprising estimates in the change in average methylation levels with age for each CpG site in a plurality of CpG sites, creating a filtered methylation profile of the single cell comprising a defined number of CpG sites that exhibit the greatest absolute Pearson correlation with an age in the reference methylation probability data set, wherein the CpG sites are those common between the single cell and the reference methylation probability dataset, calculating the likelihood of observing the filtered methylation profile of the single cell for a plurality of ages, and determining the age for which the likelihood is greatest among the ages in the plurality of ages to produce the epigenetic age of the single cell. This method, when modified, is also amenable to estimate epigenetic age from shallow methylation sequencing data in bulk samples. Altogether, this framework enables both high-resolution epigenetic age profiling in single cells, combined with drastic cost-reduction for shallow bulk epigenetic age profiling.

### CROSS-REFERENCE TO RELATED APPLICATION

This application claims benefit of U.S. Provisional Application Nos. 63/160,246 and 63/229,167,filed Mar. 12, 2021 and Aug. 4, 2021, respectively, the contents of which are incorporated herein by reference in their entirety.

### BACKGROUND OF THE INVENTION

Among several canonical hallmarks, aging involves profound epigenetic alterations, particularly at CG dinucleotides (CpGs). Changes in CpG methylation with age may be assayed using a variety of approaches, ranging from hybridization arrays to genome-wide or targeted next-generation sequencing methods. These techniques permit quantitative examination at single-base resolution of the dynamic DNA methylation landscape in any tissue of interest in organisms, such as mammals, that evolved this type of regulation.

Since their inception in the last decade, predictive multivariate machine learning models based on DNA methylation (DNAm) levels, termed ‘epigenetic clocks,’ have altered the aging field. First built strictly as an estimator of chronological age, clocks can now also integrate and predict various measures of biological aging and disease risk, underscoring their clinical relevance. Excitingly, several pan-tissue mammalian clocks were recently developed that can profile epigenetic age in virtually any tissue across eutherians with impressive precision. Epigenetic clocks are of particular interest within the scopes of lifespan extension and cell reprogramming, as these models show promise in detecting even small changes in biological age that result from these interventions.

While individual cells are the units of life, all existing epigenetic clocks rely on measurements derived from bulk samples (i.e., samples containing many cells), both for the creation and application of these models. Using bulk samples for DNA methylation analysis has been an inherent requirement of the methodologies available, which required hundreds of nanograms of input material due to harsh chemical treatment of DNA by bisulfite conversion. While bulk DNAm profiles are advantageous in that they allow for consistent and robust coverage of CpGs across the genome, the use of bulk tissue inherently obscures the epigenetic heterogeneity that exists among individual cells.

Advances in epigenomic sequencing methods have made it possible to evaluate limited methylation profiles at the single-cell level. Since the inception of these techniques in the previous decade, a variety of single-cell methylation sequencing methods have become available, including single-cell reduced representation (scRRBS) and single-cell (whole genome) bisulfite sequencing (scWGBS/scBS). These approaches rely on key modifications of the original bulk techniques that enable reduced DNA loss during library preparation. Methods for sequencing RNA, DNA methylation, and chromatin accessibility in the same single cell have also been devised, allowing for unparalleled integration of multi-omic analyses at maximal resolution.

Despite such progress, a need exists for accurate epigenetic profiling; in particular, using few cells.

### SUMMARY OF THE INVENTION

In one aspect, the invention provides a method of estimating an epigenetic age of a single cell from a mammalian tissue, the method comprising:

providing a reference methylation probability data set comprising estimates in the change in average methylation levels with age for each CpG site in a plurality of CpG sites,

providing a filtered methylation profile of the single cell comprising a defined number of CpG sites (e.g., a percentage of total sites) that exhibit the greatest absolute Pearson correlation with age in the reference methylation probability data set, wherein the CpG sites are those common between the single cell and the reference methylation probability dataset,

calculating the likelihood of observing the filtered methylation profile of the single cell for a plurality of ages, and

determining the age for which the likelihood is greatest among the ages in the plurality of ages to produce the epigenetic age of the single cell.

In some embodiments, the greatest absolute Pearson correlation is that which corresponds to at least 90^{th} (e.g., at least 91^{st}, at least 92^{nd}, at least 93^{rd}, at least 94^{th}, at least 95^{th}, at least 96^{th}, at least 97^{th}, at least 98^{th}, or at least 99^{th}; e.g., 90^{th} (e.g., 91^{st}, 92^{nd}, 93^{rd}, 94^{th}, 95^{th}, 96^{th}, 97^{th}, 98^{th}, or 99^{th}) percentile of Pearson correlation coefficients for a single cell. In some embodiments, the reference methylation probability data set comprises the estimates produced using a linear model (e.g., univariate linear model) and training data. In some embodiments, based on the univariate models and the filtered methylation profile, the posterior probability of observing unmethylated or methylated states in a single cell for any given age is computed. In some embodiments, the training data are from bulk methylation profiling (e.g., RRBS DNAm array profiling) of mammalian tissues. In some embodiments, the single cell has a sparse methylome profile. In some embodiments, the single cell has a partial methylome profile compared to the methylome profiles used in the reference methylation probability data set. In some embodiments, the step of determining comprises the use of bulk methylation data to train linear regression models that can predict methylation levels given exclusively age as the input. In some embodiments, the method further includes, using a selected fraction of age-related CpGs and their associated probabilities, calculating the likelihood that a cell comes from a tissue of a certain chronological age and registering the age of maximum likelihood as an ultimate predictor of epigenetic age. In some embodiments, the absolute Pearson correlation is at least 0.81 (e.g., at least 0.81, at least 0.82, at least 0.83, at least 0.84, or at least 0.85). In some embodiments, the reference methylation probability data set comprises 10², 10³, 10⁴, 10⁵, 10⁶, or 10⁷ or more CpG reads. In some embodiments, the filtered methylation profile comprises digitized (e.g., 0 or 1) methylation values. In some embodiments, the filtered methylation profile further comprises intermediate methylation values between 0 and 1 (e.g., 0.5). In some embodiments, the likelihood is computed for every CpG in the filtered methylation profile based on the absolute distance between the observed methylation value and the linear regression estimate at each age step within a wide range. In some embodiments,

In an aspect, the invention provides a method of estimating an epigenetic age for a low-pass sample, the method comprising:

providing a reference methylation probability data set comprising estimates in the change in average methylation levels with age for each CpG site in a plurality of CpG sites,

providing a filtered methylation profile of the low-pass sample comprising a defined number of CpG sites that exhibit the greatest absolute Pearson correlation with an age in the reference methylation probability data set, wherein the CpG sites are those common between the low-pass sample and the reference methylation probability dataset,

calculating the likelihood of observing the filtered methylation profile of the low-pass sample for a plurality of ages, and

determining the age for which the likelihood is greatest among the ages in the plurality of ages to produce the epigenetic age of the low-pass sample.

In some embodiments, the greatest absolute Pearson correlation is that which corresponds to at least 90^{th} (e.g., at least 91^{st}, at least 92^{nd}, at least 93^{rd}, at least 94^{th}, at least 95^{th}, at least 96^{th}, at least 97^{th}, at least 98^{th}, or at least 99^{th}; e.g., 90^{th} (e.g., 91^{st}, 92^{nd}, 93^{rd}, 94^{th}, 95^{th}, 96^{th}, 97^{th}, 98^{th}, or 99^{th}) percentile of Pearson correlation coefficients for a sample. In some embodiments, the reference methylation probability data set comprises the estimates produced using a univariate linear model and training data. In some embodiments, based on the univariate models and the filtered methylation profile, the posterior probability of observing unmethylated or methylated states in the low-pass sample for any given age is computed. In some embodiments, the training data are from bulk RRBS or DNAm array profiling of mammalian tissues. In some embodiments, the low-pass sample has a sparse methylome profile. In some embodiments, the low-pass sample has a partial methylome profile compared to the methylome profiles used in the reference methylation probability data set. In some embodiments, the step of determining comprises the use of bulk methylation data to train linear regression models that can predict methylation levels given exclusively age as the input. In some embodiments, the method further comprises, using a selected fraction of age-related CpGs and their associated probabilities, calculating the likelihood that the low-pass sample comes from a tissue of a certain chronological age and registering the age of maximum likelihood as a predictor of the epigenetic age. In some embodiments, the absolute Pearson correlation is at least 0.81. In some embodiments, the reference methylation probability data set comprises 10², 10³, 10⁴, 10⁵, 10⁶, or 10⁷ or more CpG reads. In some embodiments, the filtered methylation profile comprises digitized (e.g., 0 or 1) methylation values. In some embodiments, the filtered methylation profile further comprises intermediate methylation values between 0 and 1 (e.g., 0.5). In some embodiments, the likelihood is computed for every CpG in the filtered methylation profile based on the absolute distance between the observed methylation value and the linear regression estimate at each age step within a wide range.

In another aspect, the invention provides a method of estimating epigenetic age of single cells in any mammalian tissue comprising

estimating the change in average methylation levels with age for each CpG site using a univariate linear model and training data from bulk RRBS or DNAm array profiling to create a reference methylation probability dataset,

isolating common CpG sites between any given single-cell profile and the reference methylation probability dataset,

selecting a defined number (e.g., percentage) of CpGs that exhibit the greatest absolute Pearson correlation with age in the reference methylation probability dataset to create a filtered methylation profile of an individual cell,

calculating the likelihood of observing this filtered methylation profile of an individual cell at any given age, and

determining the age for which this likelihood is maximal, thereby creating an accurate epigenetic age metric in single cells with different and sparse methylome profiles.

In some embodiments, the greatest absolute Pearson correlation is that which corresponds to at least 90^{th} (e.g., at least 91^{st}, at least 92^{nd}, at least 93^{rd}, at least 94^{th}, at least 95^{th}, at least 96^{th}, at least 97^{th}, at least 98^{th}, or at least 99^{th}; e.g., 90^{th} (e.g., 91^{st}, 92^{nd}, 93^{rd}, 94^{th}, 95^{th}, 96^{th}, 97^{th}, 98^{th}, or 99^{th}) percentile of Pearson correlation coefficients for a single cell. In some embodiments, bulk methylation data is used to train linear regression models that can predict methylation levels given exclusively age as the input. In some embodiments, based on the univariate models and the filtered single cell methylation profile, the posterior probability of observing unmethylated or methylated states in a single cell for any given age is computed. In some embodiments, the method further includes, using a selected fraction of age-related CpGs and their associated probabilities, calculating the likelihood that a cell comes from a tissue of a certain chronological age and registering the age of maximum likelihood as an ultimate predictor of epigenetic age.

In yet another aspect, the invention provides a computer-readable storage medium comprising computer-readable code that, when executed by a computer, causes the computer to perform the method described herein.

In still another aspect, the invention provides use of the method described herein or the computer-readable storage medium described herein to prevent or treat disease, screen agents for retarding or accelerating aging, or assess exposure to environmental agents over time.

In a further aspect, the invention provides methods, systems, computer readable media, and compositions for single cell epigenetic age profiling as described herein.

### BRIEF DESCRIPTION OF THE DRAWINGS

FIG. 1A-1F show an overview for developing the scAge framework. FIG. 1A shows a schematic of differential read alignment in bulk (left) and single-cell (right) sequencing approaches. Single cells typically have much fewer mapped reads leading to lower overall CpG coverage. FIG. 1B shows alteration in the conventional relationship between methylation level and age at any given CpG. Instead of using methylation as a predictor of age, this approach relies on conceiving of age as a predictor of average methylation level. FIG. 1C shows quantitative linear relationships between age and methylation level are computed for every CpG in the training dataset. FIG. 1D shows CpGs from the bulk training dataset (pink) are intersected with those in single cells (blue/orange). Common CpGs between any one single cell and the bulk dataset are ranked based on correlation with age, and age-related CpGs are taken as input for scAge. FIG. 1E shows a schematic description of scAge algorithm. Age-dependent probabilities are computed based on observed methylation status in single cells. A broad likelihood metric is derived from the product of all individual probabilities. To overcome underflow errors, this metric is practically computed as the sum of log-likelihoods. FIG. 1F shows a schematic representation of the distribution of age-dependent likelihood metrics in a young (left) and old cell (right).

FIG. 2 shows that single cell sparsity prevents the application of conventional elastic net regression methods: Schematic comparison of bulk (left) and single cell (right) methylation sequencing approaches. Using bulk samples ensures high and consistent CpG coverage, while single-cell profiles typically suffer from effectively random and sparse coverage. Bulk samples produce continuous values from 0 to 1, while single cell samples exhibit primarily binarized methylation. Consequently, conventional feature tables used to train elastic net regression clocks are unfeasible to create in single cells owing to the presence of extensive missing data.

FIG. 3A-3H shows that scAge correctly recapitulates the age of hepatocytes and embryonic fibroblasts. FIG. 3A shows the progressive intersection of CpG profiles between single hepatocytes. As more profiles are intersected, random CpG coverage leads to minimal common overlap between all cells. FIG. 3B shows the mean global methylation in MEFs and young and old hepatocytes. ** denotes p<0.01. FIG. 3C shows a schematic of the liver scAge clock. FIG. 3D shows an application of the liver scAge clock on all young and old hepatocytes. FIG. 3E shows a schematic of the multi-tissue scAge clock. This predictor is composed of liver, kidney, blood, lung, muscle, and adipose tissue. FIG. 3F shows an application of the multi-tissue scAge clock on all young and old hepatocytes. FIG. 3G shows an application of the liver scAge clock on mouse embryonic fibroblasts and young/old hepatocytes. * denotes p<0.05, *** denotes p<0.001. FIG. 3H shows an application of the multi-tissue scAge clock on mouse embryonic fibroblasts and young/old hepatocytes. * denotes p<0.05, ** denotes p<0.01.

FIG. 4A-4D show single cell bisulfite sequencings results in highly variable CpG coverage between studies. FIG. 4A shows CpG coverage among MEFs, and young and old hepatocytes. MEFs exhibited significantly higher coverage compared to young and old hepatocytes. * denotes p<0.05. FIG. 4B shows CpG coverage in young and old muscle stem cells. Old muscle stem cells displayed slightly elevated CpG coverage. Cells with CpG coverage less than 1,000,000 (dotted line) were filtered out prior to downstream analysis. ** denotes p<0.01. FIG. 4C shows CpG coverage in ESCs, embryoid bodies, and oocytes. FIG. 4D shows CpG coverage in embryonic tissue. Cells were filtered to only retain those with at least 500,000 CpGs (dotted line) covered prior to downstream analysis.

FIG. 5A-5F show correlations and regressions are less prominent in multi-tissue datasets compared to liver-exclusive data. FIG. 5A shows frequency distributions of Pearson correlation coefficients for CpGs in either liver-exclusive (orange) or multi-tissue (blue) training dataset. FIG. 5B shows frequency distribution of linear regression coefficient for CpGs in either liver-exclusive (orange) or multi-tissue (blue) training dataset. FIG. 5C shows minimum and maximum Pearson correlation coefficients in either liver-exclusive (orange) or multi-tissue (blue) training dataset. FIG. 5D shows Minimum and maximum linear regression coefficients in either liver-exclusive (orange) or multi-tissue (blue) training dataset. FIG. 5E shows relationship between liver and multi-tissue correlation coefficients. FIG. 5F shows relationship between liver and multi-tissue linear regression coefficients.

FIG. 6A-6B show that scAge results with outliers removed offer improved prediction metrics on single hepatocytes. FIG. 6A shows liver scAge predictions with putative accelerated aging outliers removed (SRR3136629 and SRR3136664). FIG. 6B shows multi-tissue scAge predictions with putative accelerated aging outliers removed (SRR3136629 and SRR3136664).

FIG. 7 shows key metrics optimize with an intermediate number of CpGs considered per cell. Liver and multi-tissue scAge predictive metrics (mean absolute error, Pearson r, and Spearman rho) change when varying the number of CpGs considered per cell. Changing the number of CpGs considered from 0 to 100 in steps of 5 shows a clear improvement as additional CpGs are integrated in both hepatocyte datasets with outliers present (A) or outliers removed (B). Changing the number of CpGs considered from 0 to 10,000 in steps of 500 shows that an intermediate number of CpGs considered per cell results in the maximum predictive accuracy of the liver and multi-tissue scAge models with outliers present (C) or outliers removed (D).

FIG. 8A-8E depict stem cells showing distinct epigenetic aging patterns. FIG. 8A shows an application of the multi-tissue scAge on muscle stem cells. Predicted ages in old stem cells are significantly lower than their expected chronological age. Cells were filtered to retain only those with at least 1,000,000 CpGs covered to ensure consistent predictions. FIG. 8B shows Predicted ages depict a small but significant difference between young and old muscle stem cells. *** denotes p<0.001. FIG. 8C shows mean methylation levels in embryonic stem cells, embryoid bodies, and MII oocytes. Cells grown in 2i medium conditions exhibit significant hypomethylation compared to serum-grown ESCs. *** denotes p<0.001. FIG. 8D shows application of the liver scAge to embryonic stem cells, embryoid bodies, and MIl oocytes. Serum grown ESCs show low epigenetic age around 0, while 2i cells exhibit significantly and consistently increased epigenetic age. *** denotes p<0.001. FIG. 8E shows application of the multi-tissue scAge to embryonic stem cells, embryoid bodies, and MIl oocytes. The multi-tissue model shows more variable predictions for 2i and serum ESCs. ** denotes p<0.01, *** denotes p<0.001.

FIG. 9A-9E show epigenetic age in single cells decreases during gastrulation. FIG. 9A shows mean methylation in embryonic tissue from E4.5 to E7.5. Cells were filtered to retain only those with at least 500,000 CpGs covered. Single embryonic cells exhibit significant increased mean methylation levels from E4.5 onwards. FIG. 9B shows an application of the liver scAge clock on embryonic tissue. Cells show a consistent decreasing trend in epigenetic age, with E7.5 cells showing the lowest epigenetic age centering around 0. *** denotes p<0.001. FIG. 9C shows an application of the multi-tissue scAge clock on embryonic tissue. Cells show a similarly consistent decreasing trend in epigenetic age, although predicted ages are on average higher and more variable. *** denotes p<0.001, ** denotes p<0.01. FIG. 9D shows a schematic model of aging in single cells. Cells from the same organism show variable trajectories, with some cells aging faster (red) or slower (blue) than expected based on chronological age (green). FIG. 9E shows a schematic representation of heterogeneity within the same tissue. Despite similar cell types, tissue likely contain cells that age at different rates.

FIG. 10 shows a schematic of sub-sampling approaches. Existing bulk sample RRBS data sequenced at a high depth (>107) is randomly subsampled to a much lower depth (˜104), followed by application of the scAge framework for accurate epigenetic age profiling.

FIG. 11 shows differential methylation distributions between deep and shallow RRBS sequencing. Histogram of representative methylation levels in deep and shallow (sub-sampled to 10,000 reads) bulk RRBS data. The number of CpGs with each modality is shown. X-axis represents the methylation level, bounded in the unit interval. Y-axis represents the number of CpGs in the bin in log-scale.

FIG. 12 shows that random state does not impact CpG coverage or mean global methylation. CpG coverage (left) and mean global methylation (right) of 172 sub-sampled samples across two random seeds (“rs1” and “rs2”). No significant difference is observed between both random seeds.

FIG. 13 shows that scAge accurately tracks aging and longevity interventions in mouse blood. Epigenetic age predictions from blood in C57BL/6J mice from the Petkovich et al. study fed under a standard diet (blue) or calorically restricted (orange) across two random sub-sampling seeds (left/right).

FIG. 14 shows that scAge accurately tracks aging in Thompson et al. data. Epigenetic age predictions from blood in C57BL/6J mice from the Thompson et al. study, assessed by scAge trained on C57BL/6J blood samples from the same study.

FIG. 15 shows that scAge trained on Petkovich et al. data accurately tracks aging in Thompson et al. data. Epigenetic age predictions from blood in C57BL/6J mice from the Thompson et al. study, assessed by scAge trained on C57BL/6J blood samples from the Petkovich et al. study.

FIG. 16A-16D show a low-pass epigenetic age profiling approach. FIG. 16a shows a schematic of the analysis pipeline. Bulk samples were collected and RRBS libraries were prepared and sequenced to a high depth (>10⁷ reads). Processed methylation data was downsampled with a reproducible random seed to 10²-10⁵ reads, resulting in limited CpG profiles for each simulation. Lastly, epigenetic age (“DNAm age”) was predicted by a modified version of the scAge framework (see 16d). FIG. 16b shows a scatterplot representing the number of common CpGs after progressive intersections of regular RRBS (“Full profile”) as well as downsampled profiles (100K, 10K, and 1K CpG reads) in the Thomson et al.26 data. Individual dots show the median of 100 permutations for each intersection, with each permutation producing a randomized order of intersection. Y-axis is log-scale. Full RRBS profiles produce many intersections (blue), while downsampled profiles produce minimal to no intersections. FIG. 16c shows a barplot of the final number of common CpGs across regular RRBS (“Full”) as well as downsampled profiles (100K, 10K, and 1K CpG reads) in the Thompson et al.²⁶ data. FIG. 16d shows a scAge workflow scheme. Blood samples from young and old normal C57BL/6J mice fed with standard ad libitum diets were used to construct linear regressions in the Petkovich et al. (n=153) and Thompson et al. (n=50) data (middle and upper panels). Individual orange/purple dots schematically depict training samples, and orange/purple lines depict ordinary least squares linear regressions based on these samples. Only CpG sites that are common between a particular sample and the training dataset were considered (bottom left), and these were then filtered based on their Pearson correlation (r) with age to accommodate the desired profiling parameter (bottom right). Probability computations were performed by subtracting the distance of a particular CpG site methylation level (green) to the linear regression estimate (orange/purple) from 1 for each age step within a wide range. A final likelihood distribution (bottom middle) is then generated for each sample based on the collective probabilities harnessed across many CpGs.

FIG. 17A-17E show an application of scAge to blood data from Thompson et al. FIG. 17A show age and sex distribution of blood samples from C57BL/6J mice in the Thompson et al. dataset. Females are shown in orange, and males in blue (nₜₒₜₐₗ=50). FIGS. 17B-E show boxplots of prediction metrics on the entirety of the Thompson et al. dataset. Predictions using the Thompson et al. training dataset are shown in the left panels, and predictions harnessing regression patterns in the Petkovich et al. dataset are shown on the right. Individual dots (black) depict prediction metrics for a particular random state, number of downsampled reads, and scAge profiling parameter. Individual boxplots depict the median and the 1^{st} and 3^{rd} quartiles, with whiskers extending to 1.5× the interquartile range. Boxplots are colored based on the scAge parameter used for these predictions (i.e., the number of CpGs to profile in the likelihood computation). (FIG. 17B) depicts Pearson correlations, (FIG. 17C) depicts the associated p-value, (FIG. 17D) depicts the mean absolute error in months, and (FIG. 17E) depicts the median absolute error in months.

FIG. 18 shows epigenetic age predictions in the Thompson et al. blood dataset. Scatterplots of epigenetic age predictions for C57BL/6J blood samples in the Thompson et al. dataset (n=50). Panels on the left depict predictions using the Thompson et al. training dataset, while panels on the right show predictions using the Petkovich et al. dataset. The particular random seed used is shown in the bottom right and corresponds across training datasets. The Pearson correlation (r), the associated p-value (p), and the median absolute error (MedAE) is shown for each plot. Data shown are from the best performing set of parameters (100,000 reads, 1,000 CpGs), based on benchmarking performed (see FIG. 17).

FIG. 19A-19E show an application of scAge to blood data from Petkovich et al. FIG. 19A shows age and sex distribution of normal blood samples from C57BL/6J mice in the Petkovich et al. dataset (n=153). FIGS. 19B-19E show boxplots of prediction metrics on the entirety of the Petkovich et al. dataset. Predictions using the Thompson et al. training dataset are shown in the left panels, and predictions harnessing regression patterns in the Petkovich et al. dataset are shown on the right. Individual dots (black) depict prediction metrics for a particular random state, number of downsampled reads, and scAge profiling parameter. Individual boxplots depict the median and the 1^{st} and 3^{rd} quartiles, with whiskers extending to 1.5× the interquartile range. Boxplots are colored based on the scAge parameter used for these predictions (i.e., the number of CpGs to profile in the likelihood computation). (FIG. 19B) depicts Pearson correlations, (FIG. 19C) depicts the associated p-value, (FIG. 19D) depicts the mean absolute error in months, and (FIG. 19E) depicts the median absolute error in months.

FIG. 20 shows epigenetic age predictions in the Petkovich et al. blood dataset. Scatterplots of epigenetic age predictions for C57BL/6J blood samples in the Petkovich et al. dataset (n=153). Panels on the left depict predictions using the Thompson et al. training dataset, while panels on the right show predictions using the Petkovich et al. dataset. The particular random seed used is shown in the bottom right and corresponds across training datasets. The Pearson correlation (r), the associated p-value (p), and the median absolute error (MedAE) is shown for each plot. Data shown are from the best performing set of parameters (100,000 reads, 500 CpGs), based on benchmarking performed (see FIG. 19).

FIG. 21A-21C show a comparison of linear association metrics between the Thompson et al. and Petkovich et al. datasets. Density plots depicting the correspondence of (FIG. 21A) Pearson correlation coefficients, (FIG. 21B) linear regression coefficients, and (FIG. 21C) linear regression intercepts between training data from Petkovich et al. (x-axis) and Thompson et al. (y-axis). The number of common CpGs between both datasets is shown (n), as well as the Pearson correlation coefficient (r) and the associated p-value.

FIG. 22A-22B show consistency of epigenetic age predictions across random seeds. FIG. 22A and FIG. 22B show boxplots of inter-seed Pearson correlation (r) metrics on the entirety of (FIG. 22A) the Thompson et al. dataset and (FIG. 22B) the Petkovich et al. dataset. Inter-seed associations using the Thompson et al. training dataset are shown in the left panels, and inter-seed associations in the Petkovich et al. dataset are shown on the right. Individual dots (black) depict Pearson correlation between predicted epigenetic age across two different random seeds; since 5 random seeds were utilized in total, 10 possible combinations of random seeds are possible (n=10 dots/boxplot). Individual boxplots depict the median and the 1^{st} and 3rd quartiles, with whiskers extending to 1.5x the interquartile range. Boxplots are colored based on the scAge parameter used for these predictions (i.e., the number of CpGs to profile in the likelihood computation).

FIG. 23 shows an attenuated epigenetic aging trajectories in response to calorie restriction. Boxplots of statistical testing metrics based on delta age measurements (epigenetic age-chronological age) in n=153 standard ad libitum C57BL/6J blood samples and n=20 calorically restricted (CR) C57BL/6J blood samples from the Petkovich et al. study. Significance testing metrics using the Thompson et al. training dataset are shown in the left panels, and those using the Petkovich et al. dataset in the right. Individual dots (black) depict prediction metrics for a particular random state, number of downsampled reads, and scAge profiling parameter. Individual boxplots depict the median and the 1^{st} and 3^{rd} quartiles, with whiskers extending to 1.5× the interquartile range. Boxplots are colored based on the scAge parameter used for these predictions (i.e., the number of CpGs to profile in the likelihood computation). Upper panels depict the T statistic from Welch's t-test used to quantify statistical significance between delta age in ad libitum and calorically restricted (CR) samples, with negative values indicating lower delta age in CR samples. Lower panels depict the p-value associated to this t-test.

FIG. 24 shows decreased delta age in calorically-restricted samples. Violin plots of delta age (epigenetic age-chronological age) for ad libitum (AL, n=153, red) and calorically-restricted (CR, n=20, green) C57BL/6J blood samples in the Petkovich et al. dataset. Panels on the left depict predictions using the Thompson et al. training dataset, while panels on the right show predictions using the Petkovich et al. dataset. The particular random seed used is shown in the top right and corresponds across training datasets. The p-value depicted is derived from Welch's one-tailed t-test (assuming unequal variances). Data shown are from the best performing set of parameters (100,000 reads, 2,500 CpGs), based on benchmarking performed (see FIG. 19). Inner boxplots depict the median and the 1^{st} and 3^{rd} quartiles, with whiskers extending to 1.5× the interquartile range.

FIG. 25A-25B shows that scAge tracks epigenetic aging reversal by iPSC reprogramming. FIGS. 25A and 25Bb show boxplots of statistical testing metrics based on epigenetic age measurements in (FIG. 25A) n=3 renal fibroblasts and n=3 iPSC samples derived from these renal fibroblasts and (FIG. 25B) n=3 lung fibroblasts and n=3 iPSC samples derived from these lung fibroblasts from the Petkovich et al. study. Significance testing metrics using the Thompson et al. training dataset are shown in the left panels, and those using the Petkovich et al. dataset in the right. Individual dots (black) depict prediction metrics for a particular random state, number of downsampled reads, and scAge profiling parameter. Individual boxplots depict the median and the 1^{st} and 3^{rd} quartiles, with whiskers extending to 1.5× the interquartile range. Boxplots are colored based on the scAge parameter used for these predictions (i.e., the number of CpGs to profile in the likelihood computation). Upper plots in each panel depict the T statistic from Welch's t-test used to quantify statistical significance between epigenetic age in fibroblasts and iPSC samples, with negative values indicating lower average epigenetic age in iPSC samples. Lower panels depict the p-value associated to this t-test.

FIG. 26 shows age reversal assessed by scAge in renal fibroblasts and derived iPSCs. Box plots of epigenetic age for kidney fibroblasts (n=3, red) and kidney-derived induced pluripotent stem cells (iPSC, n=3, green) from C57BL/6J mice in the Petkovich et al. dataset. Panels on the left depict predictions using the Thompson et al. training dataset, while panels on the right show predictions using the Petkovich et al. dataset. The particular random seed used is shown in the top right and corresponds across training datasets. The p-value depicted is derived from Welch's one-tailed t-test (assuming unequal variances). Data shown are from the best performing set of parameters (100,000 reads, 500 CpGs), based on benchmarking performed (see FIG. 19). Boxplots depict the median and the 1^{st} and 3^{rd} quartiles, with whiskers extending to 1.5× the interquartile range.

FIG. 27 shows an age reversal assessed by scAge in lung fibroblasts and derived iPSCs. Box plots of epigenetic age for lung fibroblasts (n=3, red) and lung-derived induced pluripotent stem cells (iPSC, n=3, green) from C57BL/6J mice in the Petkovich et al. dataset. Panels on the left depict predictions using the Thompson et al. training dataset, while panels on the right show predictions using the Petkovich et al. dataset. The particular random seed used is shown in the top right and corresponds across training datasets. The p-value depicted is derived from Welch's one-tailed t-test (assuming unequal variances). Data shown are from the best performing set of parameters (100,000 reads, 500 CpGs), based on benchmarking performed (see FIG. 19). Boxplots depict the median and the 1^{st} and 3^{rd} quartiles, with whiskers extending to 1.5× the interquartile range.

FIG. 28A-28B show attenuated epigenetic aging from genetic interventions. FIGS. 28A-B show boxplots of statistical testing metrics based on epigenetic age measurements in (FIG. 28A) n=15 GRHKO and n=11 wildtype C57BL/6J×BALB/cByJ)/F2 blood samples of both sexes, aged 6 months and (FIG. 28B) n=8 Snell dwarf and n=10 wildtype (DW/J×C3H/HEJ)/F2 blood samples of both sexes, aged 6 months, from the Petkovich et al. study. Significance testing metrics using the Thompson et al. training dataset are shown in the left panels, and those using the Petkovich et al. dataset in the right. Individual dots (black) depict prediction metrics for a particular random state, number of downsampled reads, and scAge profiling parameter. Individual boxplots depict the median and the 1^{st} and 3^{rd} quartiles, with whiskers extending to 1.5× the interquartile range. Boxplots are colored based on the scAge parameter used for these predictions (i.e., the number of CpGs to profile in the likelihood computation). Upper plots in each panel depict the T statistic from Welch's t-test used to quantify statistical significance between epigenetic age in genetically altered and wild-type samples, with negative values indicating lower epigenetic age in GHRKO/Snell dwarf samples. Lower panels depict the p-value associated to this t-test.

FIG. 29 shows decreased delta age in GHRKO samples. Violin plots of delta age (epigenetic age−chronological age) for wild type (n=11, red) and growth hormone receptor knockout (GHRKO, n=15, green) samples from (C57BL/6J×BALB/cByJ)/F2 mice in the Petkovich et al. dataset. Panels on the left depict predictions using the Thompson et al. training dataset, while panels on the right show predictions using the Petkovich et al. dataset. The particular random seed used is shown in the top right and corresponds across training datasets. The p-value depicted is derived from Welch's one-tailed t-test (assuming unequal variances). Data shown are from the best performing set of parameters (100,000 reads, 10,000 CpGs), based on benchmarking performed (see FIG. 19). Inner boxplots depict the median and the 1^{st} and 3^{rd} quartiles, with whiskers extending to 1.5× the interquartile range.

FIG. 30 shows decreased delta age in Snell dwarf samples. Violin plots of delta age (epigenetic age−chronological age) for wild type (n=10, red) and Snell Dwarf (n=8, green) samples from (DW/J×C3H/HEJ)/F2 mice in the Petkovich et al. dataset. Panels on the left depict predictions using the Thompson et al. training dataset, while panels on the right show predictions using the Petkovich et al. dataset. The particular random seed used is shown in the top right and corresponds across training datasets. The p-value depicted is derived from Welch's one-tailed t-test (assuming unequal variances). Data shown are from the best performing set of parameters (100,000 reads, 5,000 CpGs), based on benchmarking performed (see FIG. 19). Inner boxplots depict the median and the 1^{st} and 3^{rd} quartiles, with whiskers extending to 1.5× the interquartile range.

### DESCRIPTION OF THE INVENTION

Below, we describe scAge, a statistical approach to determine the epigenetic age of single cells, and validate our results in mice. scAge tissue-specific and multi-cell type single cell clocks correctly recapitulate the chronological age of the original tissue, while uncovering the inherent heterogeneity that exists at the single-cell level. The data indicate that while cells in a tissue age in a coordinated fashion, some cells age more or less rapidly than others. We show that individual embryonic stem cells exhibit an age close to zero, that certain stem cells in a tissue show a reduced age compared to their chronological age, and that early embryogenesis is associated with the reduction of epigenetic age in individual cells, the latter supporting a natural rejuvenation event during gastrulation. scAge is both robust against the low coverage that is characteristic of single cell sequencing techniques and is flexible for studying any cell type and vertebrate organism of interest. This study demonstrates the potential for accurate epigenetic age profiling at single-cell resolution.

Our results are now described.

### I. Profiling Epigenetic Age in Single Cells

### Results

Designing scAge, a Single Cell Epigenetic Clock Framework

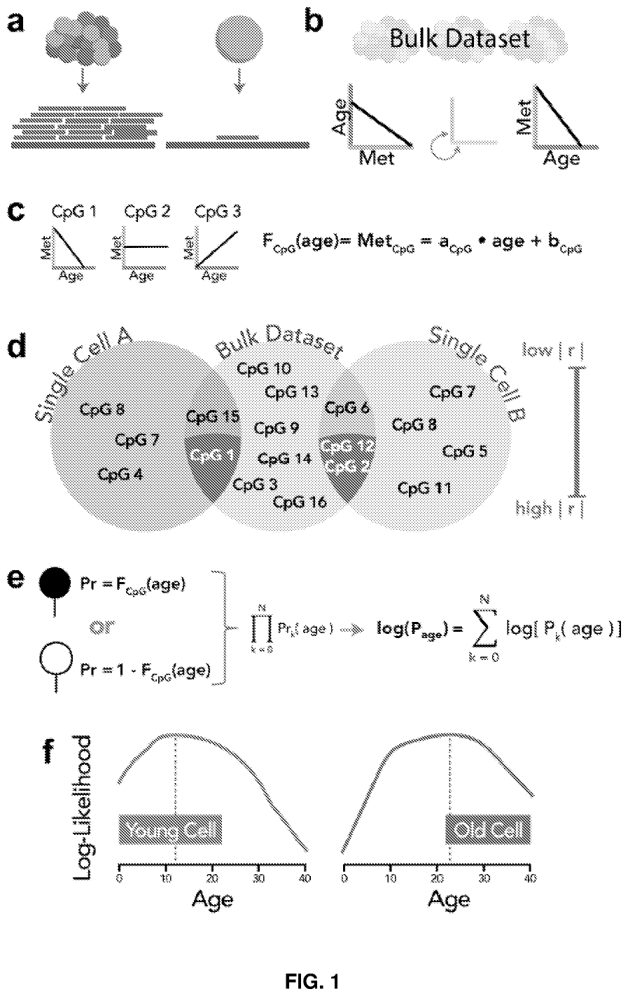

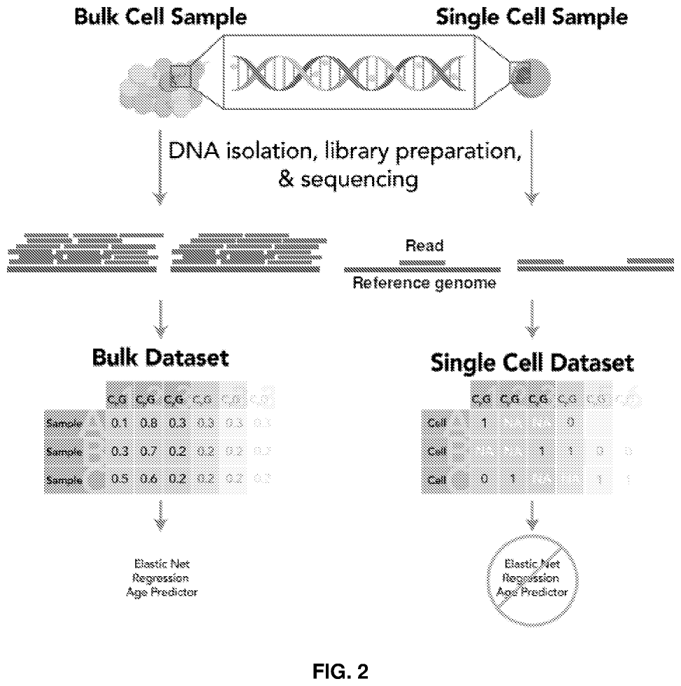

Contrary to bulk samples, sequence reads cover different parts of the genome of each single cell with very low or zero overlap among the cells (FIG. 1A, FIG. 2). To overcome these limitations, we assumed that the methylation levels of highly covered CpG sites in bulk sequencing or DNA methylation (DNAm) array profiling of a tissue offer an estimation of the probability of methylation at these particular CpG sites in any single cell coming from that tissue. Additionally, we reversed the conventional notion about the relationship between methylation level and age; while current bulk clocks use methylation level as a predictor of age, we hypothesized that age could also be thought of as a predictor of bulk methylation level at any given CpG (FIG. 1B). Using training data derived from bulk RRBS, we estimated the change in average methylation levels with age for each CpG in this reference set using a univariate linear model (FIG. 1C).

Next, we isolated the common CpG sites between any given single-cell profile and the reference methylation probability dataset (FIG. 1D). We then selected a defined number of CpGs that exhibited the greatest absolute Pearson correlation with age in the bulk data (e.g., the age-associated CpG sites). Due to the sparsity of single cell DNAm profiles, covered CpG sites vary greatly from cell to cell; despite this, a distinct collection of age-associated CpG sites are covered in every sequenced cell (FIG. 1D). In some embodiments, the greatest absolute Pearson correlation is that which corresponds to at least 90^{th} (e.g., at least 91^{st}, at least 92^{nd}, at least 93^{rd}, at least 94^{th}, at least 95^{th}, at least 96^{th}, at least 97^{th}, at least 98^{th}, or at least 99^{th}; e.g., 90^{th} (e.g., 91^{st}, 92^{nd}, 93^{rd}, 94^{th}, 95^{th}, 96^{th}, 97^{th}, 98^{th}, or 99^{th}) percentile of Pearson correlation coefficients for a single cell or for a sample.

Then, we calculated the likelihood of observing this filtered methylation profile of an individual cell at any given age (FIG. 1E). Practically, we applied logarithms (log-likelihood) to avoid underflow errors during computation. Finally, we determined the age for which this likelihood is maximal (FIG. 1F). We found that this algorithm, which we designate scAge, permitted accurate epigenetic age profiling in single cells with different and sparse methylome profiles.

### Single Cell Clock Recapitulates the Chronological Age of Single Hepatocytes

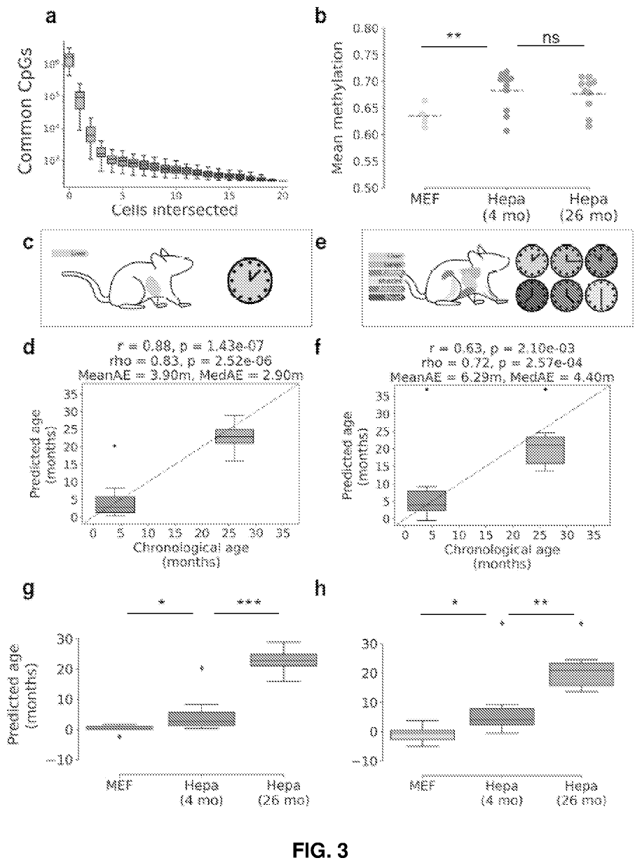

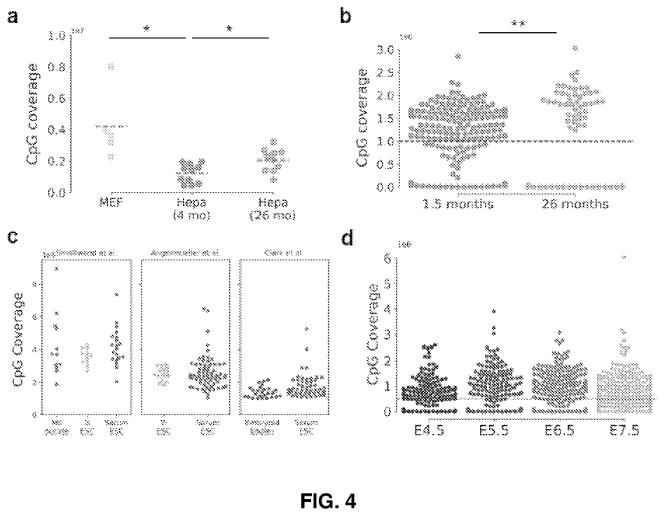

We first applied scAge to terminally differentiated cells from very young and very old mice, including 11 single hepatocytes from three 4-month-old animals and 10 single hepatocytes from 26-month-old animals (Gravina et al., Genome Biology, 17(1), 150 (2016)). Single-cell profiles contained limited common CpGs between any given pair; in fact, this effect was accentuated when sites in additional cells were progressively intersected, resulting in minimal final overlap (FIG. 3A). The coverage ranged from 0.4-3.2 million CpGs in hepatocytes, with similar mean global methylation in young and old cells (FIG. 3B, FIG. 4A). We first applied our probabilistic clock trained on bulk liver samples (Thompson et al., Aging 10, 2832-2854 (2018)) (FIG. 3C). Using only 700 independent CpGs per cell, scAge showed both impressive accuracy and consistency in age predictions for young and old hepatocytes (FIG. 3D). We achieved a Pearson r of 0.88 (Spearman rho of 0.83), with mean and median absolute errors of 3.9 and 2.9 months, respectively. Thus, liver scAge correctly recapitulated the age of the original tissue with only a handful of cells.

While scAge serves well to integrate the predictions for multiple single cells into an accurate predictor of overall tissue age, it also inherently provides increased resolution down to individual cells. Indeed, some cells were predicted to be younger or older than others in the same tissue. The smallest prediction using the liver clock for young cells was close to 0, while one cell was predicted to be around 20 months old. These results may demonstrate deep heterogeneity in the aging process, wherein global changes in epigenetic age in a bulk tissue are characterized by the uneven and diverse aging trajectories that individual cells undergo.

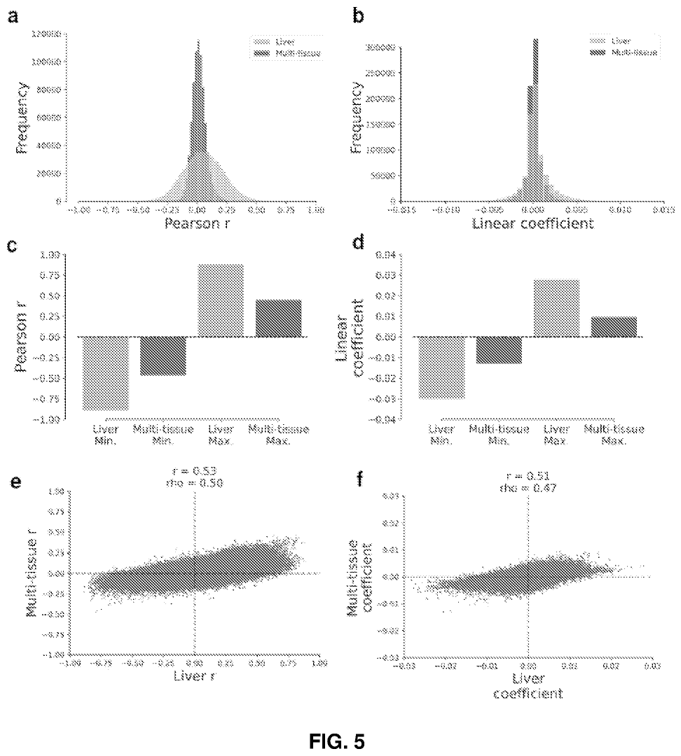

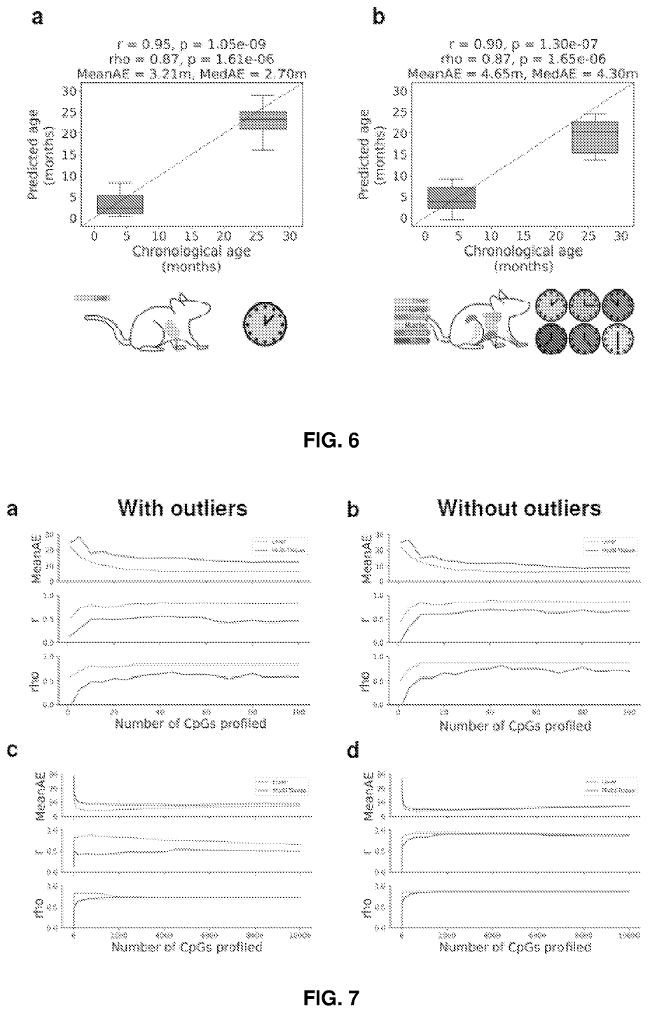

We further employed scAge trained on a multi-tissue dataset consisting of kidney, blood, liver, lung, muscle, and adipose tissue (FIG. 3E) (Thompson et al., 2018). Because multi-tissue datasets add biological noise to the relationship between age and methylation level at most CpGs, absolute correlations between both variables dropped drastically compared to a liver-exclusive dataset (FIG. 5A-F). Due to this, we reasoned that predictive metrics would improve using a multi-tissue dataset if more CpG profiles were considered per cell to compute age likelihoods. Thus, we used the multi-tissue scAge predictor with 2,000 CpGs profiled per cell. This model showed decreased accuracy compared to the liver model, with a Pearson r of 0.63 (Spearman rho=0.72) and mean and median absolute errors of 6.29 and 4.4 months, respectively (FIG. 3F). Interestingly, the multi-tissue model predicted the age of one cell in each group to be near the maximum age that we designated when running the algorithm. We interpret these observations as an accelerated aging trajectory (e.g., accelerated senescence) of some cells from the population, further underscoring the heterogeneity of epigenetic aging in single cells. Removing both outliers in the liver and multi-tissue models resulted in improved prediction accuracy, with Pearson r values of 0.95 and 0.90, respectively (FIG. 6A-B).

The predictive metrics of these two models on the liver data varied based on the number of CpGs included in the overall probability calculation, whereby incorporating too few or too many CpGs resulted in decreased prediction accuracy (FIG. 7). When too few CpGs were used, there was simply not enough data to compute a precise age prediction (FIG. 7A-B). However, the inclusion of too many CpGs also led to a decrease in the predictive accuracy of the model (FIG. 7C-D). Because our algorithm ranked CpGs based on how they are correlated with age, we suggest that including more lower-ranked CpGs introduced extensive noise into the prediction, thereby decreasing overall accuracy.

### Single Cell Clock Predicts the Age of Embryonic Fibroblasts Close to Zero

We also applied scAge to 5 mouse embryonic fibroblasts (MEFs) included in the same dataset. Of note, MEFs had significantly higher coverage compared to the hepatocytes, owing to the improved DNA quality that resulted from a milder isolation process compared to the liver cells (FIG. 4A) (Gravina et al.). Additionally, the mean methylation in MEFs was lower than that of the hepatocytes (FIG. 3B). scAge trained on either the liver or the multi-tissue datasets predicted the epigenetic age of MEFs to be around 0 (FIGS. 3G-H). Despite being in culture, these cells appeared to retain the epigenetic age information from the embryo. The predicted ages of MEFs and 4-month-old hepatocytes were nominally different using both the liver and multi-tissue models. Overall, our results showed that scAge, whether trained on a liver or multi-tissue dataset, can accurately recapitulate the chronological age of the tissue of origin in single hepatocytes and embryonic fibroblasts.

### Muscle Stem Cells Show Minimal Epigenetic Aging

To further investigate the applicability of scAge, we applied it to young and old muscle stem cell data (Hernando-Herraez et al., Nature Communications, 10(1), 4361 (2019)). This dataset consisted of 275 single cells from 6 donors, including 4 young (1.5 months) and 2 old animals (26 months). Due to technical variability in the scBS methodology, only 185 (67%) cells had greater than 1 million CpGs covered (FIG. 4B). Mean methylation between young and old cells was comparable.

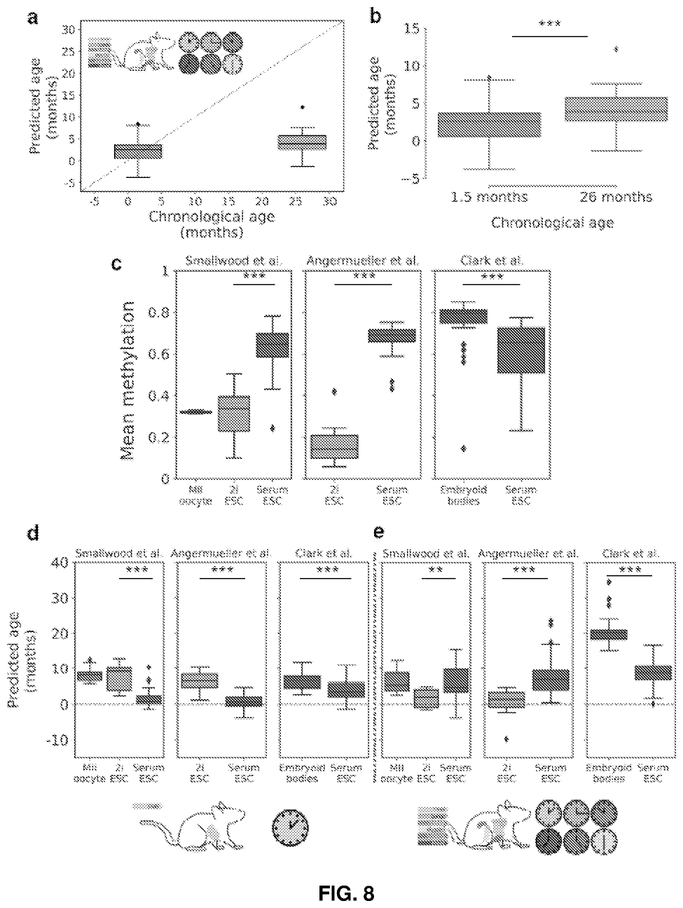

When we applied the same multi-tissue scAge that profiles 2,000 CpGs to these muscle stem cells, the epigenetic age of young cells was 9.5 weeks on average, roughly concordant with their chronological age. Interestingly, old muscle stem cells showed a significant epigenetic age increase, but only on the order of a few weeks, with a mean predicted age of 18.3 weeks (FIG. 8A-B). These results provide single-cell resolution to the data.

### Single Embryonic Stem Cells Display Low Epigenetic Age

We next sought to evaluate scAge on the most common type of publicly available single-cell methylation datasets: those profiling embryonic tissue. The epigenetic age of embryonic stem cells (ESCs) and their induced pluripotent stem cell (iPSC) counterparts generally show very low predicted epigenetic ages trending towards 0 (Horvath, Genome Biol. 14, 3156 (2013).; Meer et al., eLife 7, e40675 (2018); Petkovich et al., Cell Metab. 25, 954-960.e6 (2017)). To test our model, we examined 3 datasets of embryonic stem cells and related tissues (Angermueller et al., Nature Methods, 13(3), 229-23 (2016); Clark et al., Nature Communications, 9 (2018); Smallwood et al., Nat. Methods 11, 817-820 (2014)). Cells from these studies showed variable coverage, and we selectively filtered those that had at least 1 million CpGs covered to improve consistency (FIG. 4C). Importantly, ESCs in these studies were cultured in either traditional serum conditions, or grown in serum-free media supplemented with the “2i” cocktail of MEK and GSK3β inhibitors.

We observed significant hypomethylation among 2i cells in both the Angermueller et al. and Smallwood et al. studies (FIG. 8C). MII oocytes included in the Smallwood et al. study showed comparable mean global methylation to 2i cells, and embryoid bodies derived from ESCs in the Clark study had the greatest mean methylation on average, significantly higher than both serum-grown and 2i ESCs (FIG. 8C). We applied our liver and multi-tissue scAge models on all filtered cells from these three studies and observed consistently low predicted ages for all cell types assayed (FIG. 8D-E). Using the liver model, ESCs cultured in serum displayed an epigenetic age around 0, while 2i ESCs showed significantly higher predicted epigenetic age in both studies (FIG. 8D). However, the multi-tissue scAge predictor showed a contrasting trend (FIG. 8E).

Additionally, the multi-tissue model showed greater variance and more extreme predictions compared to the liver model in all three datasets. We suggest that this may occur due to multi-tissue datasets depicting less robust linear relationships with age, whereby the increased noise and stochasticity in methylation levels effectively translates to less consistent predictions (FIG. 5). Embryoid bodies derived from serum-grown ESCs demonstrated higher age using both clocks, hinting that the initiation of unmodulated differentiation signals into the three germ layers rapidly induces a noticeable increase in the epigenetic age of cells.

Overall, our results predicted embryonic stem cells to be close to 0 in epigenetic age, while uncovering significant differences based on culture conditions.

### Single Cell Analyses Suggest a Rejuvenation Event During Mouse Gastrulation

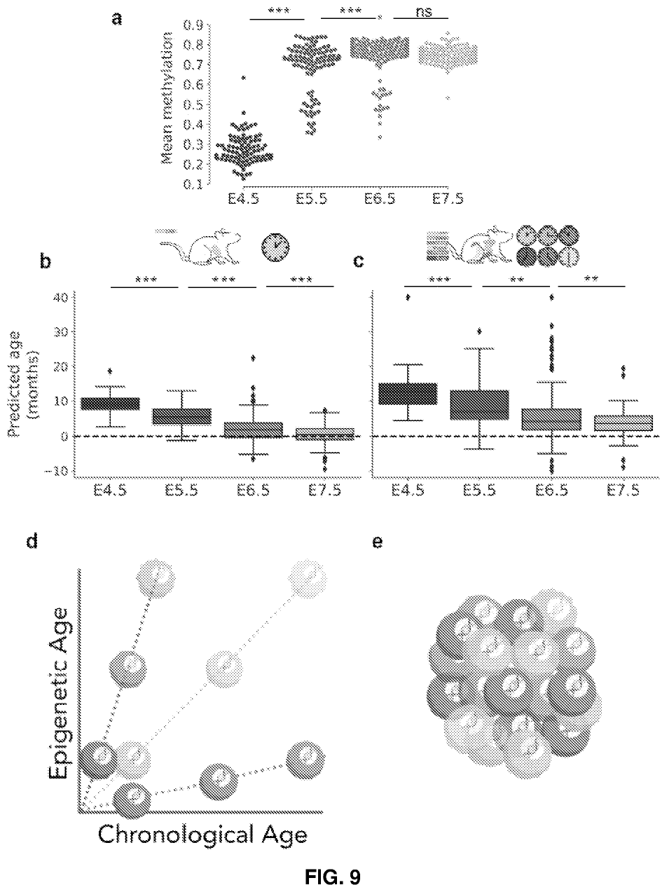

We then investigated a dataset profiling mouse gastrulation at single cell resolution (Argelaguet et al., Nature, 576(7787), 487-491 (2019)). This data consisted of 758 single cells isolated from murine embryos ranging from embryonic day (E) 4.5 to 7.5. We filtered the data for the sake of consistency to keep single cells with at least 500,000 CpGs covered, resulting in a final dataset of 495 cells (65%) (FIG. 4D). Mean global methylation varied during this early period of mouse gastrulation, with E4.5 cells showing significant hypomethylation compared to the other three developmental stages (FIG. 9A). This trend in global methylation suggests a link between ESCs grown in 2i conditions and single cells from E4.5 embryos.

Application of epigenetic clocks to bulk samples was consistent with the decrease in epigenetic age prior to ground zero. This possibility is also consistent with the understanding that damage accumulation inevitably occurs during the lifespan of an organism, even in germ cells. Thus, a rejuvenation event is thought to occur during mid-embryogenesis to ensure the continuous generation of new biologically young individuals.

To test this possibility, we applied both the liver and multi-tissue scAge clocks to single embryonic cells from the four developmental stages assayed. The liver scAge clock showed a steady and significant reduction in the mean predicted age from E4.5 to E7.5, with the latter reporting the age around 0 (FIG. 9B). The multi-tissue scAge clock showed an identical trend, with albeit slightly elevated and more variable predicted ages compared to the liver clock (FIG. 9C). Together, these results indicate that a rejuvenation event occurs during mid-embryogenesis, and points to the notion that individual cells may be rejuvenated through natural means.

In the above results, we demonstrate scAge, a statistical model to ascertain the epigenetic age of single cells. Our model utilizes bulk methylation data to train linear regression models that predict methylation levels given exclusively age as the input. Based on these univariate models, we compute the posterior probability of observing an unmethylated or methylated state in a single cell. Using a selected fraction of age-related CpGs and their associated probabilities, we calculated the likelihood that a cell comes from a tissue of a certain chronological age and registered the age of maximum likelihood as our ultimate predictor of epigenetic age. This approach solves the challenges of sparsity and uneven coverage of methylation profiles of single cells, which precluded attempts to estimate epigenetic age in individual cells. Indeed, previous epigenetic clocks require defined sets of CpG sites for their application, which is not feasible in the case of single cells.

This method enables accurate age prediction of single hepatocytes and mouse embryonic fibroblasts with high resolution on models trained either on liver or multi-tissue datasets. Additionally, we showed consistency between our model and previous work in mouse muscle stem cells, which display attenuated epigenetic aging in comparison to their chronological age. We also find that while ESCs are generally predicted to have low epigenetic age, the age differs depending on the culture condition. Finally, our data provide further evidence for the “ground zero” hypothesis of aging by showing a highly significant and steady decrease in the epigenetic age of single cells throughout the course of mouse gastrulation. Despite this advance in epigenetic age profiling in single cells, various avenues for improvement exist.

Taken together, these results suggest dramatic implications regarding epigenetic aging. We find that the aggregation of multiple single-cell predictions provides an accurate average measure of the age of a particular tissue. However, this single cell approach concurrently discovers profound heterogeneity in the aging trajectories of individual cells. This suggests that all cells in a tissue do age, but that clocks likely tick independently within single cells. In turn, some cells undergo accelerated or decelerated epigenetic aging, which was previously impossible to ascertain (FIG. 9D). This finding has applications for clinical gerontology and other areas, as it may be possible to discriminate and map “young” and “old” cells within a heterogeneous tissue via this approach (FIG. 9E).

### Methods

The above-described results were obtained using the following methods.

### Single Cell Data Processing

For the Gravina et al. study, sequence data was downloaded from the SRA under accession number SRA344045. In this case, sequence data was pre-trimmed prior to deposition to the SRA. Trimmed sequences were mapped to the mm10/GRCm38.p6 genome using Bismark v0.22.3 with the option—non_directional, as suggested by the Bismark User Guide v0.21.0 for Zymo Pico-Methyl scWGBS library preparations. Sequences were further deduplicated and methylation levels for CpG sites were extracted with Bismark (Krueger & Andrews, 2011).

For the Hernando et al., Angermueller et al., Clark et al., Smallwood et al., and Argelaguet et al. studies, processed coverage files containing extracted methylation levels generated by Bismark were downloaded directly from the GEO database under accession numbers GSE121436, GSE68642, GSE109262, GSE56879, and GSE121690, respectively (Angermueller et al., 2016; Argelaguet et al., 2019;Clark et al., 2018; Hernando-Herraez et al., 2019; Smallwood et al., 2014). A summary table of datasets analyzed, and their corresponding cell types and cell numbers is provided (Table 1).

TABLE 1
Metadata for datasets used in this study

| | | Cell types & age | Number of |
|---|---|---|---|
| Study | Year | (if applicable) | samples |
| Gravina et al. | 2016 | MEFs | 26 |
| | | 4-month hepatocytes | |
| | | 26-month hepatocytes | |
| Hernando et | 2019 | 1.5-month muSCs | 27 |
| al. | | 26-month muSCs | 5 |
| Angermueller | 2016 | 2i ESCs | 81 |
| et al. | | Serum ESCs | |
| Smallwood et | 2014 | MII oocytes | 44 |
| al. | | 2i ESCs | |
| | | Serum ESCs | |
| Clark et al. | 2018 | Embryoid bodies | 12 |
| | | Serum ESCs | 0 |
| Argelaguet et | 2019 | Early embryonic tissue | 75 |
| al. | | (E4.5, E5.5, E6.5, E7.5) | 8 |

All coverage files were then further processed to scale methylation level to a ratio between [0, 1]. While single cell methylation profiles were almost entirely binary, PCR amplification bias or other technical considerations resulted in some intermediate methylation values. Uncertain methylation calls of 0.5 were removed prior to downstream analysis. Remaining methylation values were rounded to 0 or 1. Duplicated genomic positions were also removed. Genomic positions on the 19 mouse autosomes were retained for analysis to partially minimize the effect of sex on the study. Relevant metadata were downloaded from the SRA Run Selector for all datasets.

### Bulk Data Processing

In order to create bulk reference datasets that estimate the linear relationship between age and methylation level, we downloaded processed RRBS data from the Thompson et al. study deposited in the GEO database under accession number GSE120132 (Thompson et al., 2018). This dataset consisted of 549 total samples from liver, lung, blood, kidney, adipose and muscle tissue with ages ranging from 1 month to 21 months. Methylation fractions were taken as the number of reads supporting a methylated status for a CpG over the total number of reads that covered this CpG. To maximize the accuracy of methylation levels while also preserving as many sites as possible, only CpG sites for which 90% of samples had at least 5× coverage in were retained. This resulted in a final multi-tissue matrix of 549 samples by 748,955 positive strand CpGs (autosomic chromosomes only) with some missing values. From here, a separate liver-only matrix containing 60 liver samples with ages ranging from 2 months to 20 months was created based on this same set of 748,955 CpGs.

Pearson correlations with age in months were calculated using the corrwith function from the pandas package, which automatically accounts for missing values in its processing.

Linear regressions were calculated using the LinearRegression function as part of the sklearn package. When samples contained missing CpG methylation levels, they were automatically removed along with their corresponding age prior to computing the regression equations.

### Single Cell Clock Algorithm

To devise an algorithm to ascertain epigenetic age in single cells, first, we calculated linear regressions for every CpG covered in the training dataset in the form:

$${f}_{CpG} ( Age ) = ( {Coefficient}_{CpG} * Age ) + {Intercept}_{CpG}$$

where age in months is the independent variable and f_{CpG} (Age) is the predicted methylation level for any age. We also calculated the Pearson correlation coefficient with age for every CpG in the training set. Next, we intersected the CpGs covered in the training dataset with those in any given single cell, producing a series of n CpGs that are present in both bulk and individual single-cell profiles. We subseted these n CpGs based on the absolute value of their correlation with age, selecting (in the liver model) the 700 CpGs with the largest absolute Pearson correlation and (in the multi-tissue model) the 2,000 CpGs with the largest absolute Pearson correlation. These numbers of CpGs to include in each model were determined in silico based on those that generated the most optimal accuracy metrics using the Gravina et al. dataset (FIG. 7A-D). Of note, diverse numbers of CpGs can be used with minimal fluctuations in epigenetic age predictions.

For each selected CpG per cell, we iterated through age a in steps of 0.1 months from a minimum age to a maximum age parameter. Using the linear regression formula calculated for an individual CpG, we computed f_{CpG} (age), which normally lies between 0 or 1. If this value lied outside of the range (0, 1), it was instead replaced by 0.001 or 0.999 depending on the proximity to either value. Next, we assume that the probability of observing a methylated single cell coming from a tissue of age a is approximately equal to f_{CpG}(a), that is, Pr_{CpG}(a)=f_{CpG}(a). Then, the probability that a single cell is methylated at that CpG is Pr_{CpG}(Age), and conversely the probability that a single cell is not methylated at that CpG is 1−Pr_{CpG}(Age). This provides an age-dependent probability P for every common CpG retained in the algorithm.

The product of each of these probabilities will be the overall probability of the observed methylation pattern: Pr_{Total}(a)=Πₖ₌₀ⁿPₖ(a) where k represents individual CpGs. Our goal is then to find the maximum of that product for ‘a’ (e.g., to find the most probable age for observing that particular methylation pattern). For that, we took the sum across n CpGs of the logarithm of P (to avoid running into underflow errors during computation). This gives us Σₖ₌₀ⁿ log(Pₖ(a)) for each age a. To obtain the final predicted age, we identify the age in steps of 0.1 months given a minimum and maximum age that produce the highest sum of log probabilities. This provides a likelihood metric for every age step that a single cell comes from a bulk tissue of that age. Finally, we pick the age of maximum likelihood as our predictor of age.

### Computational and Statistical Analyses

All analysis was conducted using Python 3.8.3 with the standard suite of scientific, mathematical, and plotting packages. Custom bash scripts were used to process sequencing data. Welch's t-test assuming unequal variances was used to perform all statistical tests. P-values of less than 0.05 were taken as significant. * denotes p<0.05, ** denotes p<0.01, and *** denotes p<0.001.

### II. Epigenetic Age Profiling Using Low-Pass Methylation Sequencing and a Statistical Clock Framework

In mice or other model organisms, the unavailability of methylation arrays has restricted methylation profiling to deep sequencing via reduced representation bisulfite sequencing (RRBS). In both of these approaches, there is a requirement for large amounts of input DNA, and the costs (either of the chip or the next-generation sequencing) can be prohibitively high for routine assessment of biological age. This poses a limitation for large-scale efforts to profile biological age in populations or cohorts, particularly in terms of throughput, labor, and cost.

To address this issue, we utilized scAge to profile epigenetic age in low-pass bulk bisulfite sequencing data. By utilizing a low number of sequence reads, we arrive at a mathematical modality very similar to single cells (i.e. binary methylation values of 0 or 1). Since the use of few sequence reads results in variable coverage of certain sites, our method makes use of different CpG sites in each sample for epigenetic age predictions, circumventing the need for a set of CpGs to be consistently covered across many samples (a feat that is presently accomplished using deep sequencing or methylation array analysis of bulk samples). Our approach constitutes a probabilistic algorithm that relies on tracking age-associated changes in methylation at certain CpGs and using these average changes as a probability measure. We introduce our framework as a new tool for low-cost, high-throughput epigenetic age predictions for population-scale and screening studies in model systems and in humans.

### Sub-Sampling and Differential Methylation Between Deep and Shallow RRBS

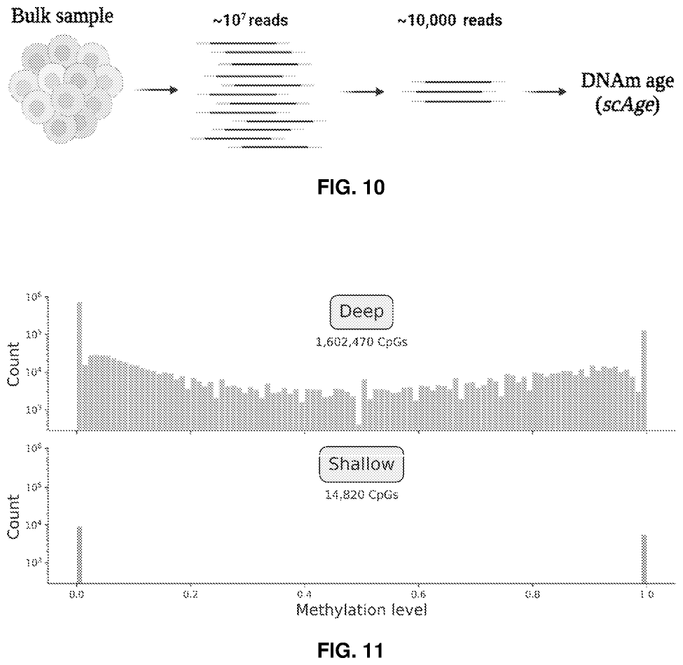

To investigate the applicability of our scAge framework in low-pass sequencing approaches, we first sub-sampled 10,000 reads from bulk blood RRBS samples from the Petkovich et al. study (Petkovich et al., Cell Metab. 25, 954-960.e6 (2017)) (FIG. 10). These samples were each originally sequenced to a depth greater than 10 million reads per sample, providing robust methylation information for 1-2 million CpGs per sample. Sub-sampling reads changed the modality of the data, converting it from fractional methylation levels to purely binary values (FIG. 11).

### Random State Does Not Impact CpG Coverage or Mean Global Methylation

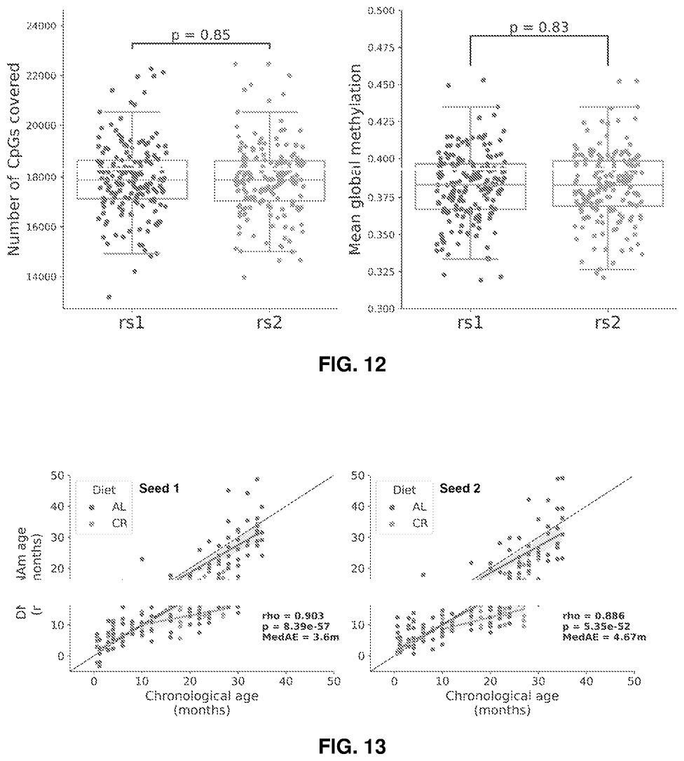

As a result of sub-sampling the data at the FASTQ file level, we obtained a distribution of CpG coverage, ranging from around 13,000 to 22,000 CpGs covered per cell. To test the reproducibility of this approach, we performed two random sub-sampling experiments, each with a different “seed” governing the random number generator. We saw no significant difference in the CpG coverage or the mean global methylation across both random samples, suggesting that the sub-sampling method is robust and produces consistent results across random states (FIG. 12). This also demonstrates that this methodology is applicable to various types of low-pass methylation data.

scAge Accurately Tracks Aging and Longevity Interventions in Mouse Blood

Next, we computed epigenetic age predictions (“DNAm age”) across 172 blood samples using our scAge framework, trained on blood data from the Petkovich et al. study. We observed a strong correlation (rho˜0.9) between chronological age and predicted age in both of the random sub-sampling experiments (FIG. 13). Thus, the age can be accurately predicted from as few as 10,000 reads instead of 10,000,000 reads per sample needed for age quantification based on standard epigenetic age profiling. This indicates that the sequencing costs may be reduced by a factor of 1000 compared to existing methods.

In addition, this method was able to discern the effect of the gold-standard longevity intervention, caloric restriction (CR), and showed a significantly decreased epigenetic age in CR samples compared to mice fed ad libitum (AL). This suggests this method is consistently robust to technical variation that may arise from sequencing, and is able to detect changes in biological aging trajectories resulting from longevity interventions.

scAge Accurately Tracks Aging in Thompson et al. Data.

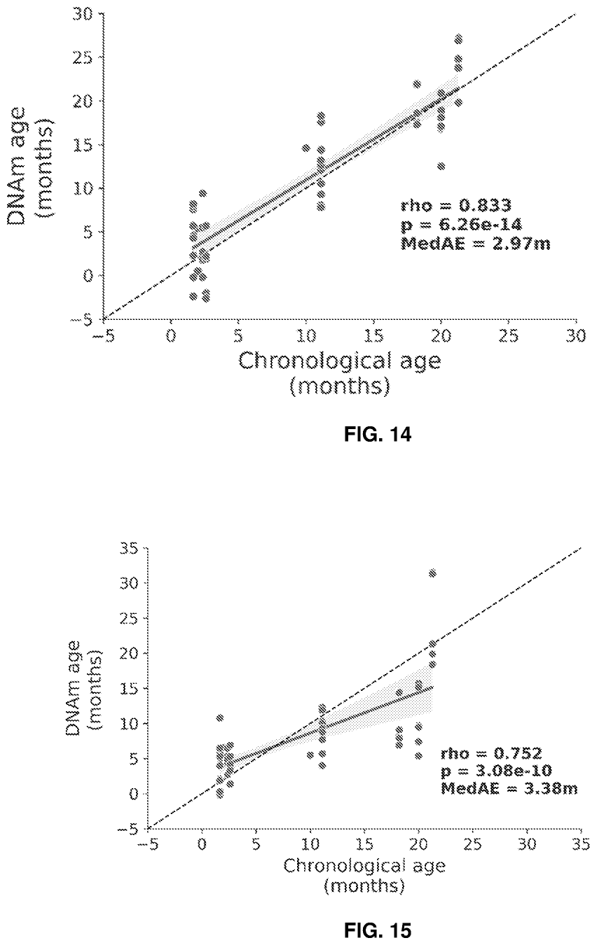

To confirm the validity of this method on an external dataset, we applied the scAge approach to profile epigenetic age in 50 C57BL/6J samples from the Thompson et al. study (Thompson et al., Aging 10, 2832-2854 (2018)²⁰ (FIG. 14). Using blood samples from this study to train our models, we obtained a strong (rho=0.83) correlation between our predicted epigenetic age in sub-sampled data and the chronological age of the animal. Additionally, our median predicted error was only ˜3 months, comparable to or lower than present approaches (Meer et al., eLife 7, e40675 (2018); Wang et al., Genome Biol. 18, 57 (2017)). This indicates that this method is highly accurate at profiling epigenetic age.

scAge Trained on Petkovich et al. Data Accurately Tracks Aging in Thompson et al. Data.

We further tested the flexibility of the method by profiling epigenetic age in the Thompson et al. blood samples with scAge trained on Petkovich et al. blood samples (FIG. 15). We observed that scAge is a robust predictor of age (rho=0.75, median absolute error=3.38 months), even when trained on data from a completely different cohort raised in a different environment. This indicates that our method is able to robustly profile age in new samples.

These above-described results demonstrate a flexible, scalable low-cost method for high-throughput profiling of epigenetic age based on low-pass (low-coverage) methylation sequencing. It allows for reducing sequencing costs by at least a factor of 1000. Our method also is useful for discerning the effect of longevity interventions, and it can be applied across tissues and species of mammals. This approach supports robust predictions based on high-throughput sequencing and multiplexing of many samples, allowing for population-scale biological age profiling, such as direct applications to consumers to assess their biological and responses to interventions and lifestyle changes, as well as clinical applications, such as analyses of large patient cohorts and biobanks. It is also useful for screening approaches, such as genetic and CRISPR screens. Overall, our framework enables inexpensive, scalable, and accurate tracking of the biological aging process, with diverse applications in the consumer, research, and clinical space.

### Methods

The above-described results were obtained using the following methods.

### Sub-Sampling Bulk RRBS Data

We downloaded raw sequence data from the SRA using sra-toolkit v2.10.8 under project accession number SRP07393016. Sequence data for C57BL/6J mice under standard diets and caloric restriction interventions were subsampled with seqtk v1.3 using reproducible random seeds to 10,000 reads/sample. Subsampled reads were trimmed using TrimGalore-0.6.6 with the options “—rrbs-paired—three_prime_clip_R1 1—clip_R1 3—three_prime_clip_R2 1—clip_R2 3” to account for sequence bias at the beginning and end of reads. Reads were aligned to the mm10/GRCm38.p6 genome using Bismark v0.22.3 in paired-end mode with standard options. Since RRBS involves targeted sequencing, reads were not deduplicated (as suggested by the Bismark User Guide v0.21.0). Methylation levels for CpGs were extracted using Bismark.

The scAge Framework

To begin, we used blood-specific methylation matrices to compute linear regression equations and Pearson correlations between methylation level and age for each CpG. These equations were in the form

$${f}_{CpG} ( age ) = Met = {m}_{CpG} * age + {b}_{CpG}$$

where age is treated as the independent variable predicting methylation, and m and b are the slope and intercept of the CpG-specific regression line, respectively. This enabled the creation of reference linear association metrics between methylation level and age for each CpG covered in the training dataset.

Next, we intersected binarized methylation profiles of low-pass samples with the reference data, producing a set of n common CpGs shared across both datasets. For each sample, we filtered these n CpGs based on the absolute value of their correlation with age, selecting the most age-associated CpGs in every sample. We denote a defined number of CpGs to profile per sample, based on the age-association ranking obtained. Alternatively, a percentile metric (top x % age-associated CpGs) or a correlation cutoff (CpGs with r>0.6) can be used to select CpGs.

For each selected CpG per sample, we iterated through age in steps of 0.1 months from a minimum age to a maximum age value. These parameters may be changed when running the algorithm to any desired resolution and range. Using the linear regression formula calculated per individual CpG in a training set, we computed the predicted methylation, f_{CpG}(age), which by the nature of the data normally lies between 0 or 1. If this predicted value was outside of the range (0, 1), it was instead replaced by 0.001 or 0.999 depending on the proximity to either value. This ensured that predicted bulk methylation values were bounded in the unit interval, corresponding to a range between fully unmethylated and fully methylated. Next, we assumed that the probability of observing a methylated value coming from a tissue of a given age was approximately equal to f_{CpG}(age), that is, Pr_{CpG}(age)=f_{CpG}(age). As an example, if a particular bulk tissue is 70% methylated (methylation=0.7), we expect that any random read from this tissue has a 70% chance of showing a methylated status for a particular CpG. Thus, the probability that a read from a sample was methylated at that CpG is Pr_{CpG}(age), and conversely the probability that a read from a sample was not methylated at that CpG is 1−Pr_{CpG}(age). This provided an age-dependent probability for every common CpG retained in the algorithm.

The product of each of these probabilities will be the overall probability of the observed methylation pattern: P(age)=Πₖ₌₁ⁿPₖ(age) where k represents individual CpGs. Our goal is then to find the maximum of that product among different ages (i.e., to find the most probable age for observing that particular methylation pattern). Practically, we compute the sum across CpGs of the natural logarithm of the individual age-dependent probabilities, preventing underflow errors when many CpGs are considered. This gave us Σₖ₌₁ⁿ ln(Pₖ(age)) for each age step. By harnessing the relationship of methylation level and age at many CpGs, these logarithmic sums ultimately provide a single likelihood metric for every age for a particular sample. Finally, we pick the age of maximum likelihood as our predictor of epigenetic age for a low-pass sample.

Additionally, using scAge on low-pass data, we obtained the following results.

### Results

### Simulating Low-Pass Bisulfite Sequencing Data

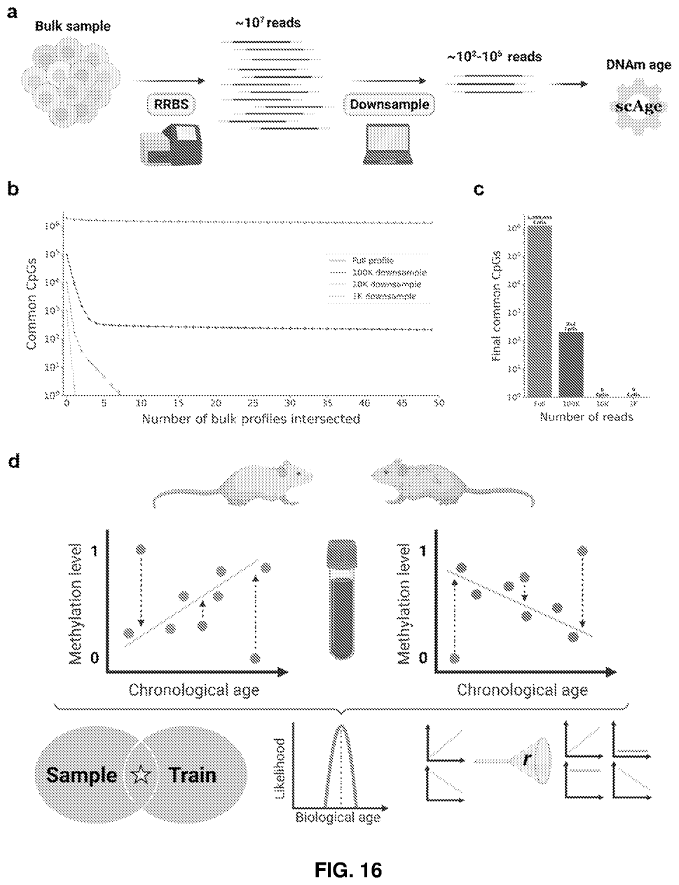

To develop an approach for prediction of epigenetic age from low-coverage bisulfite sequencing data, we first designed a simulation pipeline that enables the creation of methylation profiles from randomly downsampled sequencing reads (FIG. 16a). We utilized existing RRBS data, wherein individual libraries were sequenced to a depth greater than 10⁷ reads/sample. We next devised a random downsampling algorithm that takes as input all individual CpG reads and outputs a desired number of subsampled reads within any desired range. Practically, we downsampled bulk data to 10², 10³, 10⁴ and 10⁵ reads to assess a range of different low-pass bisulfite sequencing outputs.

Since bulk RRBS protocols enable readout at a few million CpGs per sample, downsampling to low numbers of reads (100-100,000) or performing low-pass sequencing creates a fundamental limitation for current epigenetic clock approaches. These methods traditionally rely on training machine learning models, e.g. based on elastic-net regression, which select a defined set of informative CpGs that are each assigned a weight in a resulting linear model; these weighted methylation values, adjusted by an intercept, can then be directly used for epigenetic age prediction. However, training these models requires that many CpGs are covered at high depth consistently across many samples, which can presently only be accomplished by costly deep targeted sequencing or methylation array technologies. Additionally, once a set of CpG sites is chosen and given weights in the model, the same CpG sites must all be present in any testing dataset in order to obtain the most accurate predictions. This can be circumvented to some degree with imputation approaches, but an excessive number of missing values tend to greatly reduce the absolute accuracy of current clocks. While intersecting bulk genomic RRBS data produces large (105-106 CpGs) feature tables amenable to machine learning approaches, low-pass sequencing simulations reveal that profiles have minimal common sites, precluding the use of conventional methods (FIGS. 16b, c).

To address this limitation, we applied the scAge framework, which is amenable to sparsely covered data with different sets of CpG sites available in each individual sample or cell (FIG. 16d). Briefly, the framework first trains models of methylation level based on age at many CpGs. Next, we employ a selective intersection algorithm: only CpG sites common to a sample/cell of interest and the training data are retained, and these are subsequently filtered to keep only highly age-associated CpGs sites. We then cycle through each CpG site in the profile, and measure the distance between the observed methylation value and the linear model estimate at a particular age. We treat this distance as a probability metric, meaning that ages with smaller absolute distance between the observed value and the training model are more probable for a particular sample. Lastly, we harness these individual CpG-specific probabilities into a broader likelihood profile, which enables epigenetic age profiling given sparse, data.

We adjusted the original scAge algorithm to conform to the notion that while the binary modality in single-cell data is reflective of inherent biology in most cell types, bulk samples (even downsampled ones) may exhibit meaningful non-binary methylation, given that sequencing reads from many cells are obtained during library preparation. Altogether, we developed a pipeline that can take as input real or simulated low-pass data for epigenetic age predictions using scAge.

As training data to the framework, we used C57BL/6J blood samples from the Petkovich et al. (n=153) and Thompson et al. (n=50) studies. Samples ranged in age from 1 month to 35 months in the Petkovich et al. data, and 2 to 21 months in the Thompson et al. data. We selected highly covered (depth≥5×) CpG sites in each sample and constructed a large feature table for all samples per dataset across all identified CpGs. To ensure enough valid samples per CpG were present for each subsequent linear regression computation, we dropped CpG sites for which more than 10% of samples had missing values. Ultimately, this generated two independent training datasets: 1) 1.2 million CpG linear regressions in the Thompson et al. blood data and 2) 1.9 million CpG linear regressions in the Petkovich et al. blood data. Of note, CpG sites in the Thompson et al. data were concatenated to the positive strand. This, combined with the different number of samples in each dataset, accounts for the disparity in the number of CpG linear models per dataset.

scAge Can Track the Aging Process in Blood Based on Low-Pass Sequencing Data

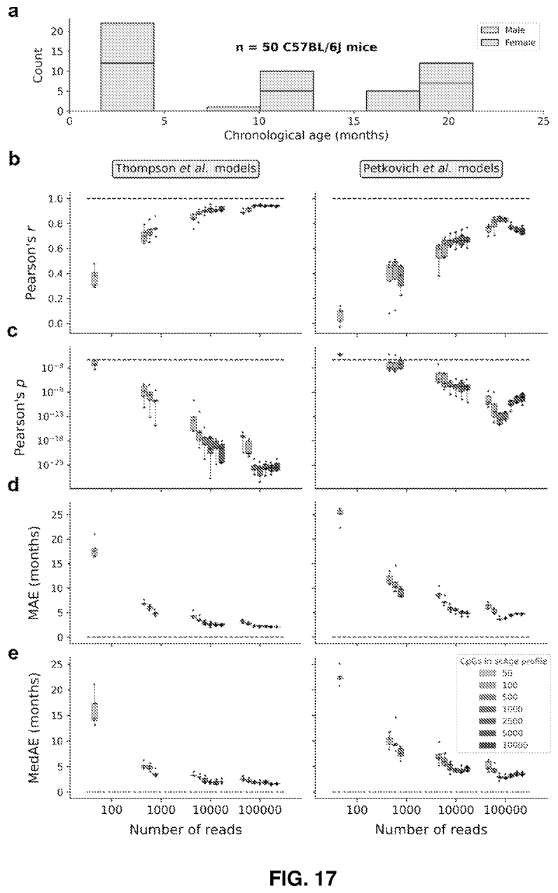

We first trialed our approach on downsampled data from the Thompson et al. study (FIG. 17a). We employed 10², 10³, 10⁴ and 10⁵ CpG reads across 5 different random seed; this enabled randomization of the set of CpGs in the subsample, and mirrors picking random small subsets of bisulfite sequencing libraries. For each downsampled random set of CpG matrices, we applied our modified scAge framework trained on all samples from either the Petkovich et al. or Thompson et al. data, profiling a variety of fixed CpG numbers ranging from 50 to 104 (FIG. 17b-e).

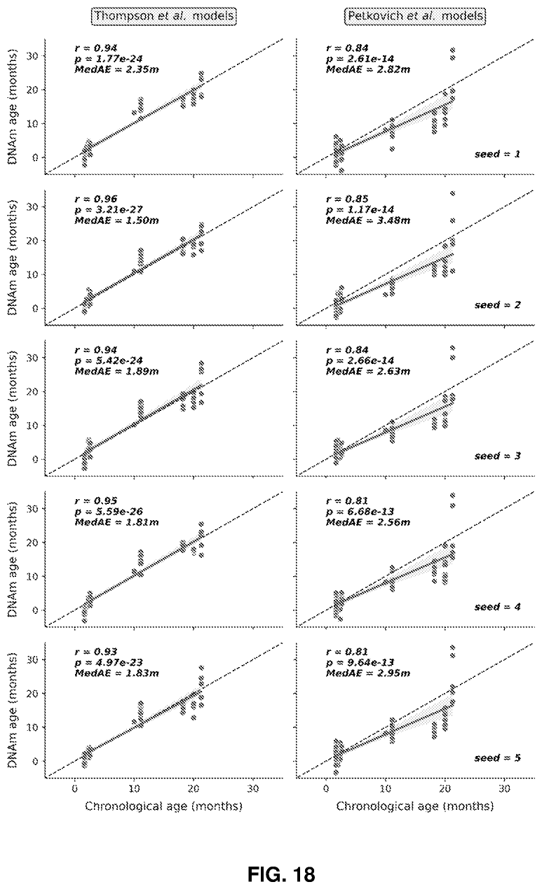

We observed robust performance of our epigenetic age profiling approach in these data, with the results varying both based on the number of reads in the downsampled data and the number of age-associated CpGs included in the scAge likelihood profile. Pearson correlations generally increased in magnitude and significance as more reads were subsampled, with some variation based on the size of the likelihood profile (FIGS. 17b, c). Mean and median absolute errors decreased using both models as the number of reads was increased, again with some variation based on scAge profile sizes (FIGS. 17d, e). Using these benchmarking results, we selected the best performing parameters according to both training datasets (100,000 reads, 1,000 CpGs profiled by scAge/sample). When Thompson et al. linear regressions were applied, correlation coefficients ranged from 0.93-0.96 depending on the random seed (FIG. 18). The independent Petkovich et al. models showed slightly decreased predictive accuracy, with Pearson correlations ranging from 0.81-0.85. Median errors were low with both training datasets, ranging from 1.81-2.35 months with Thompson et al. training and 2.56-3.48 months with Petkovich et al. training across random seeds. Interestingly, predictive metrics were almost equally strong when only 10,000 reads per sample were assayed (FIG. 17). This suggests that integration of the scAge framework and low-pass sequencing with a relatively small number of reads may be sufficient to accurately measure epigenetic age.

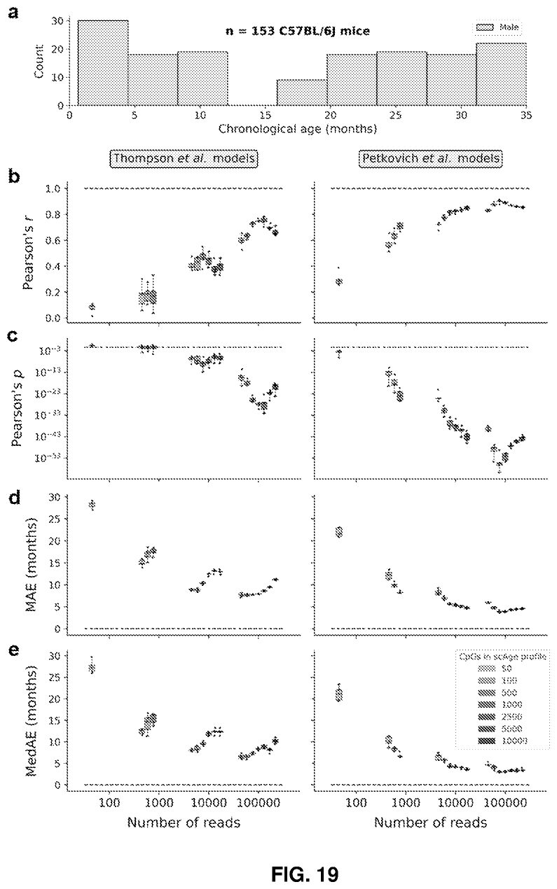

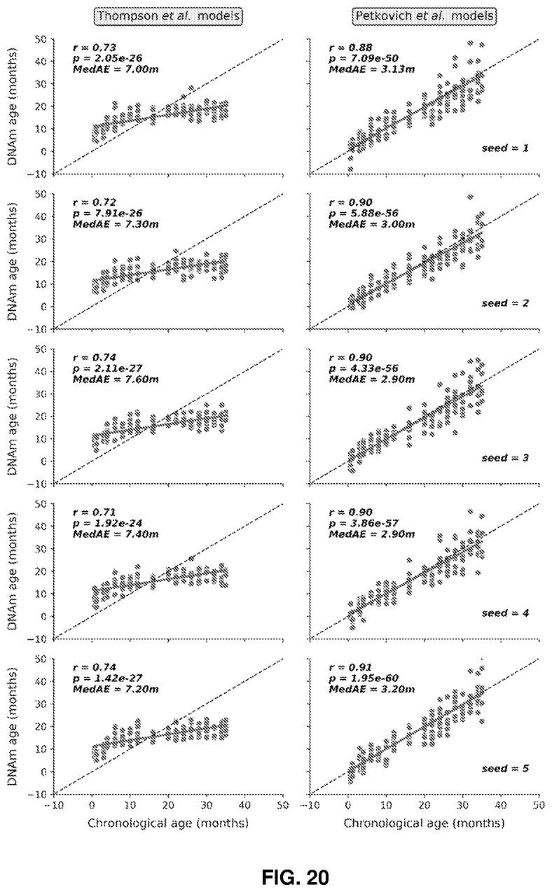

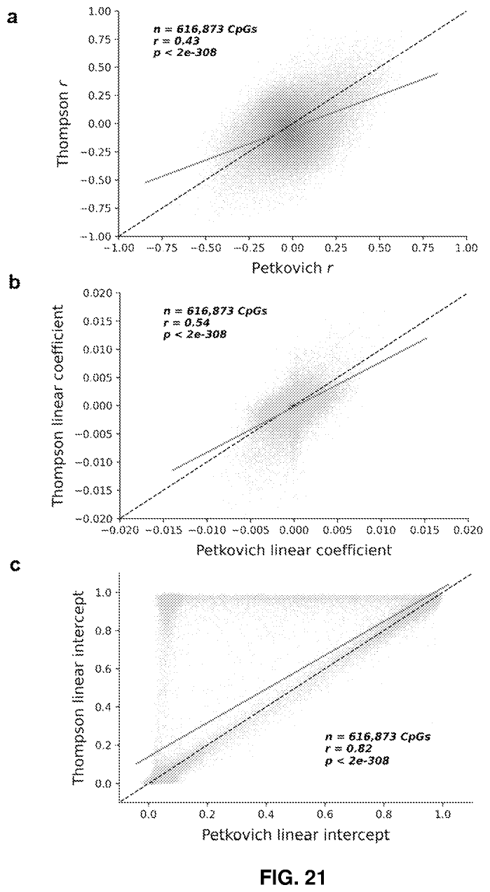

Next, we applied the same methodology on downsampled data from the Petkovich et al. study, which included 153 blood samples from standard male C57BL/6J mice aged 1-35 months (FIG. 19a). Again, we observed good performance of both training datasets on these data, with similar parameter trends as to what we observed upon application to downsampled Thompson et al. data (FIG. 19b-e). A greater number of reads was associated with improved prediction accuracy across all metrics, and results varied based on the number of CpGs used by scAge. When selecting the most accurate parameters based on benchmarking (100,000 reads with 500 CpGs/scAge profile), we observed Pearson correlations ranging from 0.88-0.91 and median absolute errors between 2.9-3.2 months using the Petkovich et al. model, while the Thompson model was noticeably less accurate (r=0.71-0.74, MedAE=7.0-7.6m) (FIG. 20). This may be due to the differential processing of the Thompson et al. methylation data as compared to the Petkovich et al. data, and could also be reflective of batch effects. Indeed, we observed only a moderate positive association between Pearson correlation (r=0.43) or linear regression coefficients (r=0.54) in the Petkovich et al and Thompson et al. training datasets (FIG. 21). scAge applied to low-pass data similarly had good performance when only 10,000 reads were subsampled, suggesting only a small number of reads are needed for accurate profiling.

### Consistency of Low-Pass Simulation Predictions Hint at Reproducibility of Epigenetic Age Profiling

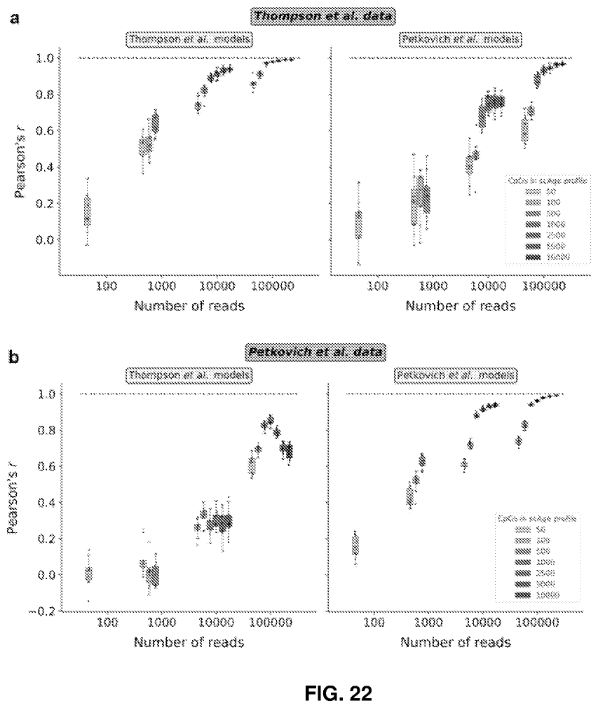

In order to best simulate different sequencing runs of the same sample, we applied a random downsampling approach that returned different sets of CpGs depending on the chosen seed. This procedure is essentially analogous to picking a random aliquot of a library for sequencing, and enables tracking the consistency of predictions from different subsamples of a particular library. While prediction accuracy based on chronological age was robust across different random seeds (FIGS. 17, 19), we were curious whether the same sample, downsampled to produce distinct CpG methylation matrices, would provide relatively similar epigenetic age readouts, mimicking resequencing of the same sample. Excitingly, the best performing tests (10,000-100,000 reads) showed strong inter-seed prediction consistency (FIG. 22). Both Thompson et al. and Petkovich et al. models had inter-seed Pearson correlations around ˜0.9 based on the Thompson et al. data. The Petkovich et al. model similarly showed robust inter-seed prediction correlations when applied to Petkovich et al. data, while the Thompson et al. models applied to this same dataset had weaker inter-seed correlations. Overall, these results suggest that our approach may be able to generate reproducible predictions from different small assortments of covered CpGs.

Low-Pass scAge Tracks Attenuated Epigenetic Aging by Caloric Restriction

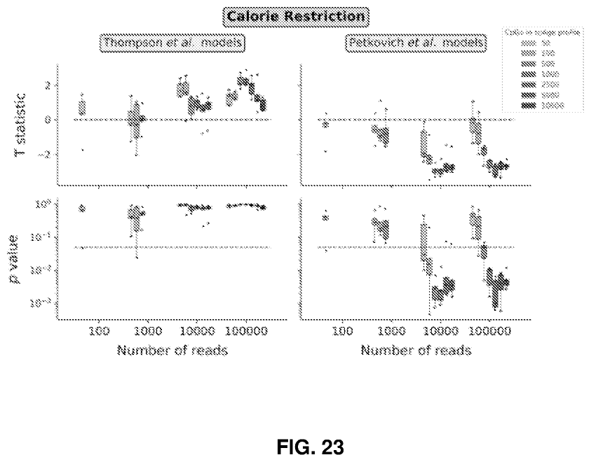

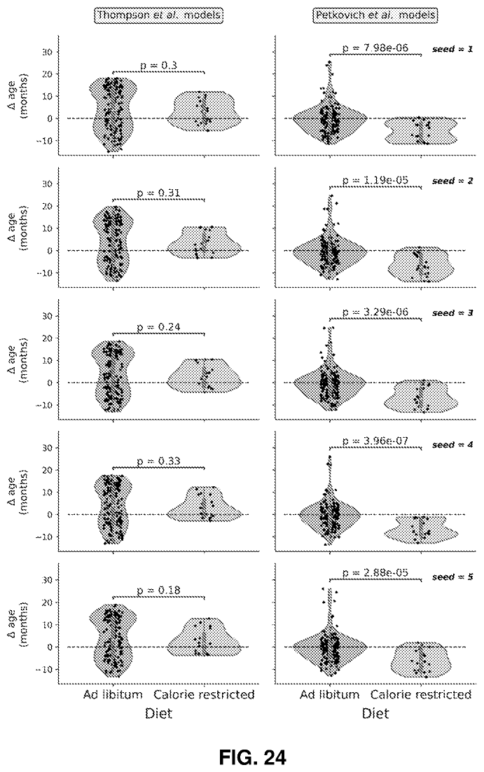

Next, we were interested if our low-pass scAge framework could distinguish the effect of calorie restriction as a longevity intervention. Caloric restriction was previously found to attenuate epigenetic aging as measured in blood and liver samples, and is commonly considered one of the gold-standard lifespan-extending interventions currently in use in model systems. To test the effectiveness of our approach, we analyzed 20 blood samples from calorie-restricted C57BL/6J mice with chronological ages ranging from 10 to 27 months. Interestingly, the Petkovich et al. model was able to reliably detect a decrease in delta age (epigenetic age minus chronological age) between control and calorically restricted mice when 10,000 or 100,000 reads were utilized (FIG. 23).

In the best performing model based on benchmarking (100,000 reads, 2,500 CpGs/profile), delta age in calorically restricted mice was significantly lower than in mice fed ad libitum across all random seeds (FIG. 24). This suggests that our method is capable of discerning the effect of longevity interventions on biological aging with few sequencing reads. However, application of the Thompson et al. models did not reveal a significant effect. We hypothesize this is likely due to the impaired accuracy of the Thompson et al. model on the Petkovich et al. data as a whole (FIG. 19b-e), which again may be caused by the alternate processing by the original authors or batch effect differences (FIG. 21).

Low-Pass scAge Identifies Age Reversal Effect by iPSC Reprogramming

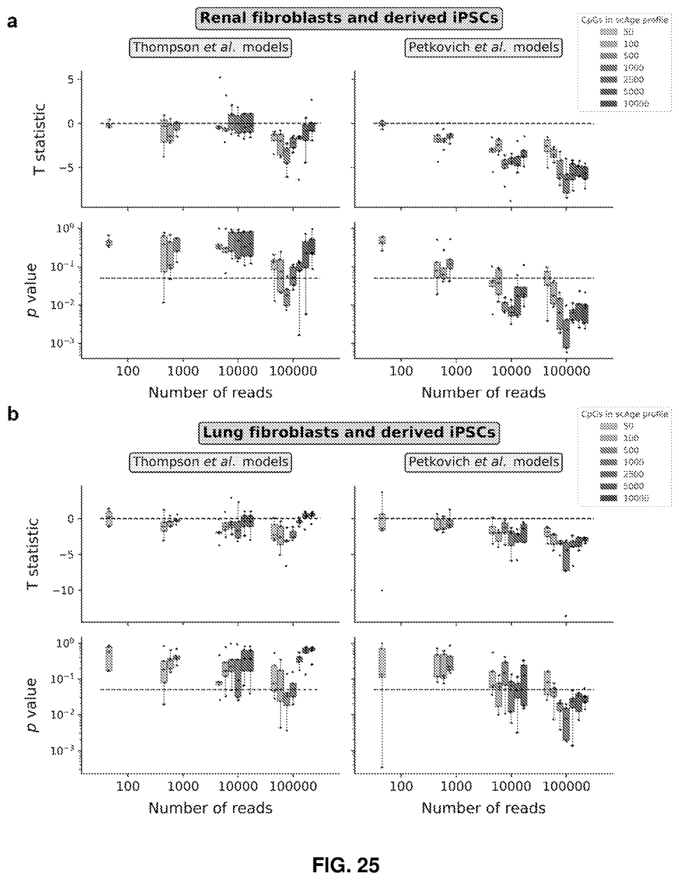

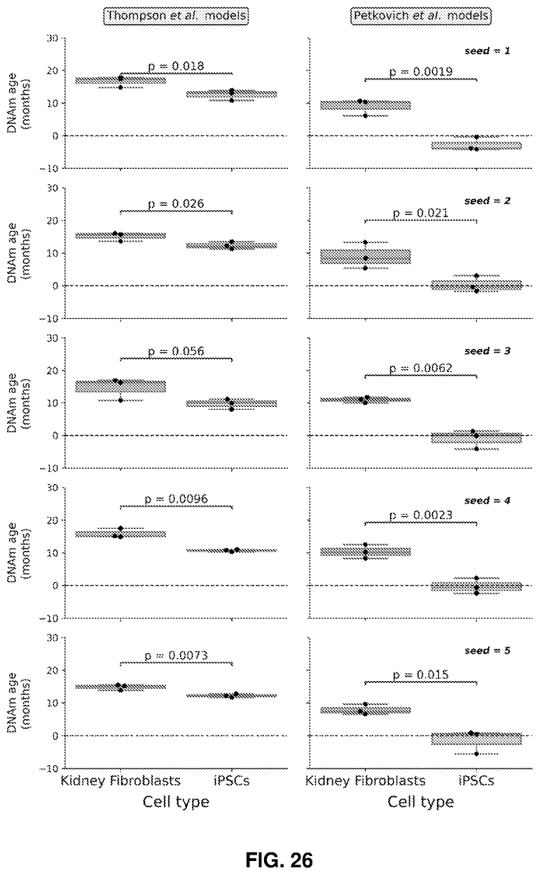

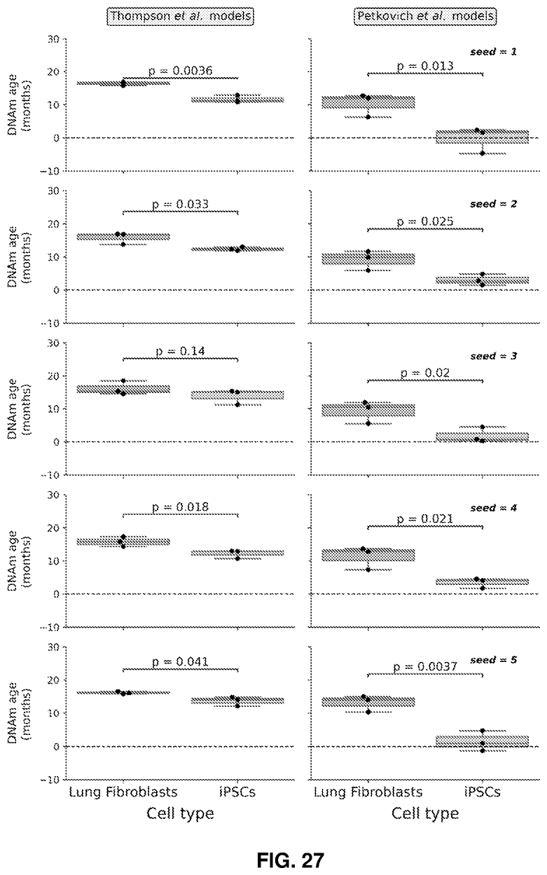

Given the rising interest in cellular reprogramming approaches for rejuvenation research, we were interested if our low-pass approach, combined with the scAge framework, could identify a significant epigenetic age decrease resulting from iPSC reprogramming. We applied our method, trained on blood methylation data, to renal and lung fibroblasts and corresponding iPSC lines derived from these tissues. We observed that predicted epigenetic ages based on Petkovich et al. linear regression models were significantly lower for iPSCs than for fibroblasts across all random seeds when using 100,000 reads and at least 500 CpG sites in each scAge profile (FIG. 25). When selecting the best model based on benchmarking (100,000 reads, 500 CpGs/scAge profile), we witnessed significant decreases in epigenetic age across random seeds using either the Petkovich et al. or Thompson et al. training data, in both kidney-derived and lung-derived iPSCs (FIGS. 26, 27). Overall, absolute age predictions had improved accuracy under Petkovich et al. models, with iPSC samples displaying epigenetic age near 0, mirroring earlier results.

These data indicate that epigenetic age reversal by Yamanaka factor-based induction of pluripotency can be assayed using low-pass approaches in combination with our statistical clock framework, inviting further applications in robustly assessing the effect of rejuvenation interventions in a high-throughput, low-cost manner.

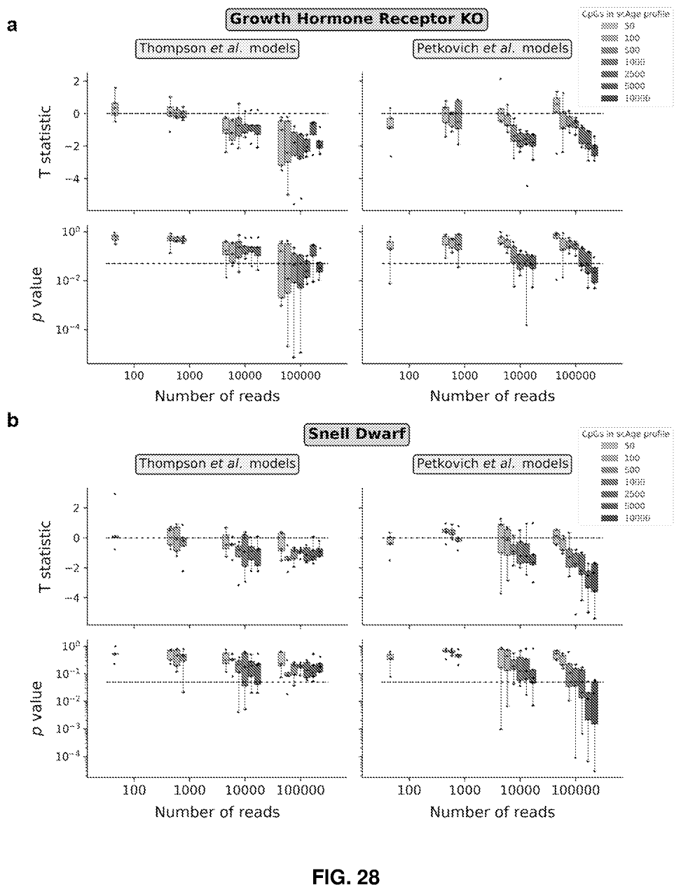

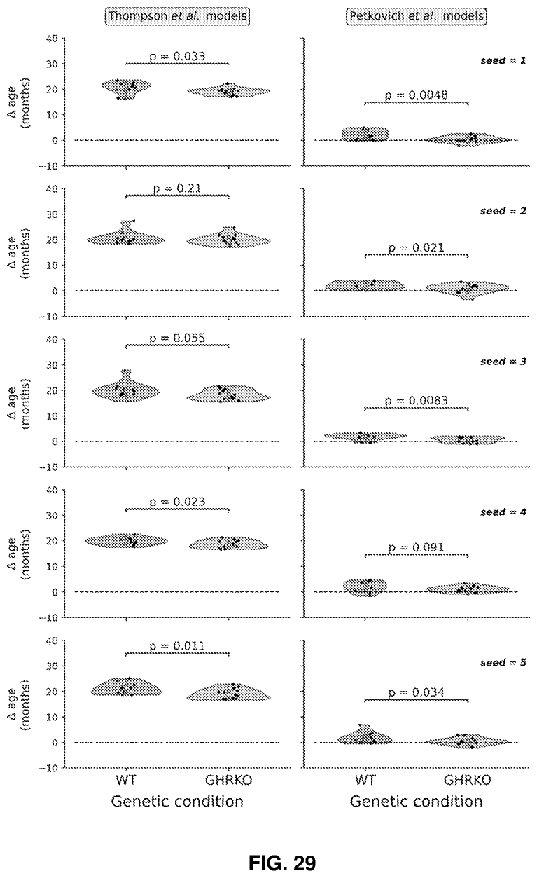

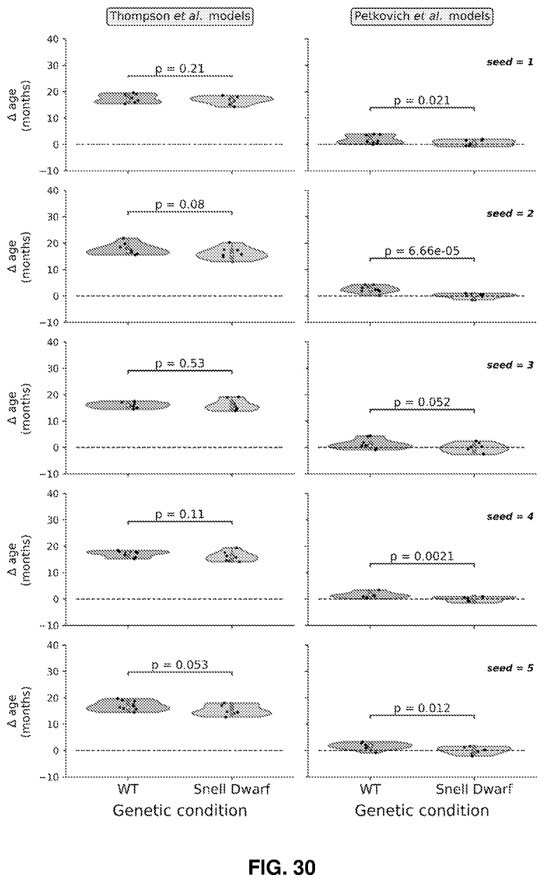

Low-Pass scAge Identifies Genetically-Based Attenuated Aging Patterns We were interested to test if the integration of low-pass sequencing approaches with our scAge framework enables assessment of attenuated biological aging derived from genetic alterations. Some genetic interventions, notably growth hormone receptor knockouts (GHRKO) and Pit1 loss-of-function mutations (Snell dwarf), have previously shown lifespan-extending effects as well as decreased biological age as assessed by a blood epigenetic clock (Petkovich et al., Cell Metab. 25, 954-960.e6 (2017); Flurkey et al., Proc. Natl. Acad. Sci. 98, 6736-6741 (2001); Coschigano et al., Endocrinology 144, 3799-3810 (2003)). We tested our approach on 6-month-old GHRKO, Snell dwarf, and control mice, and observed a small but clear trend in age reduction, particularly with the Petkovich et al. model at 100,000 reads (FIG. 28). The Thompson et al. model showed some nominal significance on GHRKO mice when 100,000 reads were downsampled, but this depended on the particular random state. By selecting the best performing model based on benchmarking (100,000 reads, 10,000 CpGs/scAge profile), we observed significant decreases in delta age (epigenetic age minus chronological age) for GHRKO mice across both training datasets in almost all random states, while the attenuated aging phenotype in Snell dwarf mice could only be significantly picked up by the Petkovich et al training dataset (FIGS. 29, 30). This is unsurprising, as some previous clocks have also failed to pick up statistically significant differences in Snell dwarf models (Meer et al., eLife 7, e40675 (2018)).

Altogether, we provide evidence that our approach discerns the attenuated epigenetic effect brought on by some genetic manipulations. Low-pass sequencing combined with our novel approach may open avenues for large-scale genetic screening applications in model systems and humans.

We report the application of a statistical clock framework, scAge, to low-pass (low coverage) bulk RRBS sequencing data, revealing robust performance at age prediction across two independent murine blood datasets (FIGS. 16, 17, 19). We randomly downsampled existing RRBS data to a defined number of reads per sample, followed by epigenetic age prediction based on the scAge approach. Predictions were stronger when the linear model training dataset used was the same as the downsampled dataset, but independent datasets still performed well, suggesting that our approach should be generalizable to new collections of data.

We showed that scAge combined with low-pass sequencing is amenable to reproducible profiling of biological age, especially when more reads are subsampled (FIG. 22). Our approach also enables tracking the attenuated biological aging effect brought on by calorie restriction (FIG. 23), as well as age reversal by induction of pluripotency through the Yamanaka factors in fibroblast lines derived from two tissues (FIG. 25). However, we observe the most significant results in the case where predictions are based on training data sourced from the same dataset as the downsampled data. Generation of additional bulk RRBS datasets amenable for training may shed light on this phenomenon and enable universal application of our framework to other datasets. Additionally, we show that some genetic interventions, namely GHRKO and Snell dwarf models, reduce biological age as predicted by scAge, but only under some parameters (FIG. 28).

Overall, we introduce the application of our recently developed scAge epigenetic age profiling framework, which relies on leveraging individual methylation-age trajectories at CpGs in deeply sequenced bulk data and applying them to shallow sequencing data. We assessed shallow data across a variety of read sets ranging from 100 to 100,000, revealing robust performance of our approach especially when 10,000-100,000 reads are used. We show that parameters of scAge have some impact on prediction metrics, although comparatively less than the number of reads sampled. Excitingly, the scAge approach combined with shallow sequencing data enables robust prediction of chronological age from as a little as 10,000 reads in standard blood samples of C57BL/6J mice across two independent datasets. Additionally, we report that our approach may be amenable to identify the effects of some longevity and rejuvenation interventions, such as calorie restriction or iPSC reprogramming, on biological aging trajectories. This suggests our method may be capable of validating current and identifying new longevity interventions.

### Methods

The above-described results were obtained using the following methods.

### Downsampling Sequencing Data

Processed methylation data were obtained from the supplementary files on the GEO database for both Petkovich et al. and Thompson et al. datasets, with accession numbers GSE8067210 and GSE12013226, respectively. Metadata was also downloaded from the GEO database using the GEOparse package implemented in Python. Methylation sequences were originally mapped to the mm10/GRCm38 mouse genome, and methylation information was extracted with Bismark36 in the case of the Petkovich et al. data, and BS-Seeker 237 in the case of the Thompson et al. data. We further filtered methylation data to include only CpGs on autosomic chromosomes, in order to partially mitigate the effect of sex on predictions. Individual CpG reads (whether methylated or unmethylated) were concatenated into lists in a randomized order to prevent any location bias from affecting downstream predictions. We selected a defined number of CpG reads ranging from 100 to 100,000, each covering mostly unique CpGs with some overlap when a larger number of reads were sampled. Methylation at subsampled CpGs was calculated as the mean of all methylation reads for that CpG in a particular subsample. This produced mostly binary methylation values (0, unmethylated; 1, methylated), with some additional values in between this range when multiple reads covered a particular CpG. In order to produce random subsamples, we used the random.sample function with different seeds (set with random.seed), generating distinct assortments of CpGs for the same sample depending on the seed.

Training scAge Models

To create linear regressions enabling epigenetic age profiling via the scAge algorithm, we utilized deeply sequenced training data from the Petkovich et al. and Thompson et al. datasets. Specifically, we filtered for only standard blood C57BL/6J samples, resulting in dataframes with n=153 and n=50 samples for the Petkovich et al.and Thompson et al. studies. For each individual sample, only CpG sites with 5× or more coverage were retained, while remaining CpG sites were marked as missing. This filtering enabled high resolution estimation of true methylation proportions in a particular sample, which is an integral component of the scAge training framework. Next, we progressively intersected all samples with an “outer” join methodology, capturing all CpGs covered 5× or more in at least one sample in the dataset. In order to select only CpGs which were deeply covered consistently across samples (enabling accurate linear regression modeling), we removed CpG sites for which more than 10% of samples had missing values.

By applying these various filtration methods to both maximize CpG coverage and methylation value accuracy, we arrived at a dataframe of 1,918,766 CpGs across 153 samples in the Petkovich et al.¹⁰ dataset and 1,202,751 CpGs across 50 samples in the Thompson et al. dataset. Notably, the Petkovich et al. dataset contained both negative and positive strand CpGs, while the Thompson et al. dataset contained only positive strand CpGs. This is a result of the processing methods used, whereby Thompson et al. concatenated positive and negative strand reads to the positive strand to increase confidence in the methylation values while decreasing the total feature space. This difference in processing may partially help to explain the deviations that we observe when comparing predictions based on each training model.

Application of the scAge Prediction Framework

To assess epigenetic age in low-pass bulk data, we could not utilize conventional mouse methylation clocks, nor could we train novel methylation clocks based on elastic net regression machine learning approaches. This is entirely due to the primary limitation of downsampled data: the lack of consistent CpG coverage across samples. In order to overcome this fundamental constraint, we made use of the recently developed scAge framework, which enables accurate epigenetic age profiling in single cells. Single cells feature notoriously sparse, binary methylation profiles as a function of current limitations in sequencing protocols, which heavily resemble downsampled bulk RRBS data. However, while the binary nature of single-cell methylation profiles is consistent with the biology underlying this data, bulk methylation profiles often contain discrete methylation values within the unit region, ideally representing the proportion of cells in a specific sample that are methylated at a specific cytosine.

Practically, downsampled data were first intersected on an individual basis with a particular training dataset. Next, of the common CpG sites between any subsample and the training set, a defined number of the most age-associated CpG sites (based on the magnitude of Pearson correlations) was chosen and varied as a parameter. This enabled testing different combinations of reads, CpGs included in the scAge profile, and random downsampling states. Among selected, highly age-associated CpG sites, the distance between the observed methylation value and the linear regression estimate for a particular CpG site and age was treated as a probability measure. In essence, we assumed the closer the observed methylation value was to the linear regression estimate for a particular age, the higher the probability of observing this methylation state at this age. Hence, we subtracted the distance between observed value and linear estimate from 1, then proceeded to take the natural logarithm of the resulting value to circumvent underflow errors in downstream processing. Once all CpG sites were assayed for a particular age, we summed log probabilities together for the entire profile (mathematically identical to taking the product of all probabilities), generating a single value describing the likelihood of observing a particular CpG profile. An age-likelihood distribution was created between −20 months and 60 months, in intervals of 0.1 months, enabling prediction of epigenetic age based on maximum likelihood estimation.

### Computational and Statistical Analyses

All analyses were performed using Python 3.9.2. Figures were generated using matplotlib 3.4.1 in combination with seaborn 0.11.1. Welch's one-tailed t-test assuming unequal variances, implemented in statannot 0.2.3 and scipy 1.6.3, was used to perform statistical tests between groups. Pearson correlations and associated p-values were computed using the pearsonr function implemented in the scipy package. Linear regressions underlying the scAge framework were computed with the LinearRegression function implemented in the scikit-learn 0.24.2 package.

### OTHER EMBODIMENTS

All publications, patents, and patent applications mentioned in this specification are incorporated herein by reference to the same extent as if each independent publication or patent application was specifically and individually indicated to be incorporated by reference.

The invention includes the following numbered paragraphs.

1. A method of estimating epigenetic age of single cells in any mammalian tissue comprising

estimating the change in average methylation levels with age for each CpG site using a univariate linear model and training data from bulk RRBS or DNAm array profiling to create a reference methylation probability dataset;

isolating common CpG sites between any given single-cell profile and the reference methylation probability dataset;

selecting a defined number of CpGs that exhibit the greatest absolute Pearson correlation with age in the reference methylation probability dataset to create a filtered methylation profile of an individual cell;

calculating the likelihood of observing this filtered methylation profile of an individual cell at any given age; and

determining the age for which this likelihood is maximal, thereby creating an accurate epigenetic age metric in single cells with different and sparse methylome profiles.

2. The method of paragraph 1 wherein bulk methylation data is used to train linear regression models capable of predicting methylation levels given exclusively age as the input.
3. The method of paragraph 2 in which, based on the univariate models and the filtered single cell methylation profile, the posterior probability of observing unmethylated or methylated states in a single cell for any given age is computed.
4. The method of paragraph 3 additionally comprising, using a selected fraction of age-related CpGs and their associated probabilities, calculating the likelihood that a cell comes from a tissue of a certain chronological age and registering the age of maximum likelihood as an ultimate predictor of epigenetic age.
5. A computer-readable medium comprising computer-readable code that, when executed by a computer, causes the computer to perform the operations set forth in paragraphs 1-4.
6. Use of the method of paragraphs 1-4 or the computer-readable medium of paragraph 5 to prevent or treat disease, screen agent for retarding or accelerating aging, or assess exposure to environmental agents over time.
7. Methods, systems, computer readable media, and compositions enabling single cell epigenetic age profiling.
8. A flexible, scalable framework for estimating the biological age of organisms from low-pass (using, for example, low coverage) bulk methylation sequencing obtained from a mammalian tissue.
9. The method of paragraph 8, wherein models are trained to estimate methylation trajectories with age at individual CpGs.
10. The method of paragraph 8, wherein CpG profiles of samples of interest are intersected with a training dataset.
11. The method of paragraph 8, wherein intersected profiles are filtered based upon the absolute correlation with age of particular CpG sites.
12. The method of paragraph 8, wherein individual probabilities are computed for every CpG in the filtered profile based on the absolute distance between the observed methylation value and the linear regression estimate at each age step within a wide range.
13. The method of paragraph 8, wherein natural logs of individual probabilities are summed together to create a broad age-likelihood profile.
14. The method of paragraph 8, wherein the age of maximum likelihood is designated the epigenetic age of the sample.
15. The method of paragraph 8, wherein decreases in biological age resulting from longevity interventions (such as caloric restriction) and reprogramming approaches (such as iPSC reprogramming) can be reliably assessed by our approach.
16. The method of paragraph 8, wherein high-throughput, low-cost epigenetic age profiling can be made available to human consumers, research scientists, and clinical professionals for routine and large-scale epigenetic aging studies.
17. The method of paragraph 8, wherein low-cost, high-throughput aging studies can be conducted in mice and humans to assess biological aging in response to existing and putative interventions.
20. A flexible, scalable method for estimating the biological age of organisms from low-pass (low coverage) methylation sequencing approaches across tissues and mammalian species.
21 The method of paragraph 20, wherein decreases in biological age resulting from longevity interventions, lifestyle changes, genetic manipulations and reprogramming approaches can be reliably assessed by our approach.
22 The method of paragraph 20, wherein high-throughput, low-cost epigenetic age profiling can be made available to human consumers, research scientists, and clinical professionals.

While the invention has been described in connection with specific embodiments thereof, it will be understood that it is capable of further modifications and this application is intended to cover any variations, uses, or adaptations described herein following, in general, the principles described herein and including such departures from the invention that come within known or customary practice within the art to which the invention pertains and may be applied to the essential features hereinbefore set forth, and follows in the scope of the claims.

Other embodiments are within the claims.

What is claimed is:

1. A method of estimating an epigenetic age of a single cell from a mammalian tissue, the method comprising:
providing a reference methylation probability data set comprising estimates in the change in average methylation levels with age for each CpG site in a plurality of CpG sites,
providing a filtered methylation profile of the single cell comprising a defined number of CpG sites that exhibit the greatest absolute Pearson correlation with an age in the reference methylation probability data set, wherein the CpG sites are those common between the single cell and the reference methylation probability dataset,
calculating the likelihood of observing the filtered methylation profile of the single cell for a plurality of ages, and
determining the age for which the likelihood is greatest among the ages in the plurality of ages to produce the epigenetic age of the single cell.

2. The method of claim 1, wherein the reference methylation probability data set comprises the estimates produced using a univariate linear model and training data.

3. The method of claim 2, wherein, based on the univariate models and the filtered methylation profile, the posterior probability of observing unmethylated or methylated states in a single cell for any given age is computed.

4. The method of claim 2 or 3, wherein the training data are from bulk RRBS, WGBS, or DNAm array profiling of mammalian tissues.

5. The method of any one of claims 1 to 4, wherein the single cell has a sparse methylome profile.

6. The method of any one of claims 1 to 5, wherein the single cell has a partial methylome profile compared to the methylome profiles used in the reference methylation probability data set.

7. The method of any one of claims 1 to 6, wherein the step of determining comprises the use of bulk methylation data to train linear regression models that can predict methylation levels given exclusively age as the input.

8. The method of any one of claims 1 to 7, further comprising, using a selected fraction of age-related CpGs and their associated probabilities, calculating the likelihood that a cell comes from a tissue of a certain chronological age and registering the age of maximum likelihood as an ultimate predictor of epigenetic age.

9. The method of any one of claims 1 to 8, wherein the absolute Pearson correlation is at least 0.81.

10. The method of any one of claims 1 to 9, wherein the reference methylation probability data set comprises 10², 10³, 10⁴, 10⁵, 10⁶, or 10⁷ or more CpG reads.

11. The method of any one of claims 1 to 10, wherein the filtered methylation profile comprises digitized methylation values.

12. The method of any one of claims 1 to 11, wherein the likelihood is computed for every CpG in the filtered methylation profile based on the absolute distance between the observed methylation value and the linear regression estimate at each age step within a wide range.

13. A method of estimating an epigenetic age for a low-pass sample, the method comprising:
providing a reference methylation probability data set comprising estimates in the change in average methylation levels with age for each CpG site in a plurality of CpG sites,
providing a filtered methylation profile of the low-pass sample comprising a defined number of CpG sites that exhibit the greatest absolute Pearson correlation with an age in the reference methylation probability data set, wherein the CpG sites are those common between the low-pass sample and the reference methylation probability dataset,
calculating the likelihood of observing the filtered methylation profile of the low-pass sample for a plurality of ages, and
determining the age for which the likelihood is greatest among the ages in the plurality of ages to produce the epigenetic age of the low-pass sample.

14. The method of claim 13, wherein the reference methylation probability data set comprises the estimates produced using a univariate linear model and training data.

15. The method of claim 14, wherein, based on the univariate models and the filtered methylation profile, the posterior probability of observing unmethylated or methylated states in the low-pass sample for any given age is computed.

16. The method of claim 14 or 15, wherein the training data are from bulk RRBS, WGBS, or DNAm array profiling of mammalian tissues.

17. The method of any one of claims 13 to 16, wherein the low-pass sample has a sparse methylome profile.

18. The method of any one of claims 13 to 17, wherein the low-pass sample has a partial methylome profile compared to the methylome profiles used in the reference methylation probability data set.

19. The method of any one of claims 13 to 18, wherein the step of determining comprises the use of bulk methylation data to train linear regression models that can predict methylation levels given exclusively age as the input.

20. The method of any one of claims 13 to 19, further comprising, using a selected fraction of age-related CpGs and their associated probabilities, calculating the likelihood that the low-pass sample comes from a tissue of a certain chronological age and registering the age of maximum likelihood as a predictor of the epigenetic age.

21. The method of any one of claims 13 to 20, wherein the absolute Pearson correlation is at least 0.81.

22. The method of any one of claims 13 to 21, wherein the reference methylation probability data set comprises 10², 10³, 10⁴, 10⁵, 10⁶, or 10⁷ or more CpG reads.

23. The method of any one of claims 13 to 22, wherein the filtered methylation profile comprises digitized methylation values.

24. The method of any one of claims 13 to 23, wherein the likelihood is computed for every CpG in the filtered methylation profile based on the absolute distance between the observed methylation value and the linear regression estimate at each age step within a wide range.

25. A method of estimating epigenetic age of single cells in any mammalian tissue comprising estimating the change in average methylation levels with age for each CpG site using a univariate linear model and training data from bulk RRBS or DNAm array profiling to create a reference methylation probability dataset,
isolating common CpG sites between any given single-cell profile and the reference methylation probability dataset,
selecting a defined number of CpGs that exhibit the greatest absolute Pearson correlation with age in the reference methylation probability dataset to create a filtered methylation profile of an individual cell,
calculating the likelihood of observing this filtered methylation profile of an individual cell at any given age, and
determining the age for which this likelihood is maximal, thereby creating an accurate epigenetic age metric in single cells with different and sparse methylome profiles.

26. The method of claim 25, wherein bulk methylation data is used to train linear regression models that can predict methylation levels given exclusively age as the input.

27. The method of claim 26, wherein, based on the univariate models and the filtered single cell methylation profile, the posterior probability of observing unmethylated or methylated states in a single cell for any given age is computed.

28. The method of claim 27, further comprising, using a selected fraction of age-related CpGs and their associated probabilities, calculating the likelihood that a cell comes from a tissue of a certain chronological age and registering the age of maximum likelihood as an ultimate predictor of epigenetic age.

29. A computer-readable storage medium comprising computer-readable code that, when executed by a computer, causes the computer to perform the method of any one of claims 1-28.

30. Use of the method of any one of claims 1-28 or the computer-readable storage medium of claim 29 to prevent or treat disease, screen agent for retarding or accelerating aging, or assess exposure to environmental agents over time.

31. Methods, systems, computer readable media, and compositions for single cell epigenetic age profiling as described herein.
